SEQUENCE LISTING (39 sequences)

SEQ ID NO: 1 - protein, 21 aa, Artificial Sequence
  Description of artificial sequence artificially synthesized sequence
MALPVTALLLPLALLLHAARP

SEQ ID NO: 2 - protein, 247 aa, Artificial Sequence
  Description of artificial sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 3 - protein, 45 aa, Artificial Sequence
  Description of artificial sequence artificially synthesized sequence
TTTPAPRPPTPAPTIASQPLSLRPEACRPAAGGAVHTRGLDFACD

SEQ ID NO: 4 - protein, 24 aa, Artificial Sequence
  Description of artificial sequence artificially synthesized sequence
IYIWAPLAGTCGVLLLSLVITLYC

SEQ ID NO: 5 - protein, 27 aa, Artificial Sequence
  Description of artificial sequence artificially synthesized sequence
FWVLVVVGGVLACYSLLVTVAFIIFWV

SEQ ID NO: 6 - protein, 42 aa, Artificial Sequence
  Description of artificial sequence artificially synthesized sequence
KRGRKKLLYIFKQPFMRPVQTTQEEDGCSCRFPEEEEGGCEL

SEQ ID NO: 7 - protein, 41 aa, Artificial Sequence
  Description of artificial sequence artificially synthesized sequence
RSKRSRLLHSDYMNMTPRRPGPTRKHYQPYAPPRDFAAYRS

SEQ ID NO: 8 - protein, 113 aa, Artificial Sequence
  Description of artificial sequence artificially synthesized sequence
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 261 aa, Artificial Sequence
  Description of artificial sequence artificially synthesized sequence
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 10 - protein, 118 aa, Artificial Sequence
  Description of artificial sequence artificially synthesized sequence
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSS

SEQ ID NO: 11 - DNA, 354 bp, Artificial Sequence
  Description of artificial sequence artificially synthesized sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagc

SEQ ID NO: 12 - protein, 114 aa, Artificial Sequence
  Description of artificial sequence artificially synthesized sequence
DIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 13 - DNA, 342 bp, Artificial Sequence
  Description of artificial sequence artificially synthesized sequence
gacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgg

SEQ ID NO: 14 - protein, 6 aa, Artificial Sequence
  Description of artificial sequence artificially synthesized sequence
SGYNWH

SEQ ID NO: 15 - protein, 16 aa, Artificial Sequence
  Description of artificial sequence artificially synthesized sequence
YIHYTGSTNYNPALRS

SEQ ID NO: 16 - protein, 9 aa, Artificial Sequence
  Description of artificial sequence artificially synthesized sequence
IYNGNSFPY

SEQ ID NO: 17 - protein, 17 aa, Artificial Sequence
  Description of artificial sequence artificially synthesized sequence
KSSQSLFNSGNQKNYLT

SEQ ID NO: 18 - protein, 7 aa, Artificial Sequence
  Description of artificial sequence artificially synthesized sequence
WASTRES

SEQ ID NO: 19 - protein, 9 aa, Artificial Sequence
  Description of artificial sequence artificially synthesized sequence
QNAYSFPYT

SEQ ID NO: 20 - protein, 20 aa, Artificial Sequence
  Description of artificial sequence artificially synthesized sequence
CTGAGCAGCGTGACCGCCGC

SEQ ID NO: 21 - protein, 20 aa, Artificial Sequence
  Description of artificial sequence artificially synthesized sequence
TGGAGTGGATCGGCTACATC

SEQ ID NO: 22 - protein, 20 aa, Artificial Sequence
  Description of artificial sequence artificially synthesized sequence
AGTAGTAGATGGCGGTGTCG

SEQ ID NO: 23 - protein, 247 aa, Artificial Sequence
  Description of artificial sequence artificially synthesized sequence
QVQLQQSGAELARPGASVKMSCKASGYTFTSYTMHWVKQRPGQGLEWIGYINPSSGYTNYNQKFKDKATLTADKSSSTAYMQLSSLTSEDSAVYYCARIYYGNSFAYWGQGTTVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKR

SEQ ID NO: 24 - protein, 242 aa, Artificial Sequence
  Description of artificial sequence artificially synthesized sequence
QVQLQQSGPELVKPGALVKISCKASGYTFTSYDINWVKQRPGQGLEWIGWIYPGDGSTKYNEKFKGKATLTADKSSSTAYMQLSSLTSENSAVYFCARGGYRYDEAMDYWGQGTTVTVSSGGGGSGGGGSGGGGSQIVLTQSPAIMSASPGEKVTMTCSASSSISYMHWYQQKPGTSPKRWIYDTSKLASGVPARFSGSGSGTSYSLTISSMEAEDAATYYCHQRSSYPYTFGGGTKLEIKR

SEQ ID NO: 25 - protein, 247 aa, Artificial Sequence
  Description of artificial sequence artificially synthesized sequence
QIQLVQSGPELKKPGETVKISCKASGYTFTNYGMNWVKQAPGKGLKWMGWINTNTGEPTYAEEFKGRFAFSLETSASTAYLQINNLKNEDTATYFCARFSYGNSFAYWGQGTTVTVSSGGGGSGGGGSGGGGSDIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPLTFGAGTKLELKR

SEQ ID NO: 26 - protein, 247 aa, Artificial Sequence
  Description of artificial sequence artificially synthesized sequence
DVQLQESGPDLVKPSQSLSLTCTVTGYSITSGYNWHWIRQFPGNKMEWMGYIHYTGSTNYNPSLRSRISITRDTSKNQFFLQLNSVTTDDTATYYCTRIYNGNSFPYWGQGTSVTVSSGGGGSGGGGSGGGGSDIVMTQSPSSLTVTPGEKVTMTCKSSQSLFNSGNQKNYLTWYQQRPGQPPKMLIYWASTRESGVPDRFTGSGSGTDFTLTISSVQAEDLAVFYCQNAYSFPYTFGGGTKLEIKR

SEQ ID NO: 27 - protein, 247 aa, Artificial Sequence
  Description of artificial sequence artificially synthesized sequence
QVQLQQSGAELARPGASVKMSCKASGYTFTSYTMHWVKQRPGQGLEWIGYIDPSSGYTNYNQKFKDKATLTADKSSSTAYMQLSSLTSEDSAVYYCARIYYGNSFAYWGQGTTVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKR

SEQ ID NO: 28 - protein, 247 aa, Artificial Sequence
  Description of artificial sequence artificially synthesized sequence
QVQLQQSGAELARPGASVKMSCKASGYTFTSYTMHWVKQRPGQGLEWIGYINPASGYTNYNQKFKDKATLTADKSSSTAYMQLSSLTSEDSAVYYCARIYYGNSFAYWGQGTTVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKR

SEQ ID NO: 29 - protein, 247 aa, Artificial Sequence
  Description of artificial sequence artificially synthesized sequence
QVQLVQSGAEVKKPGASVKVSCKASGYTFTSYTMHWVRQAPGQGLEWMGYINPASGYTNYNQKFKDRVTMTRDTSTSTAYMELSSLRSEDTAVYYCARIYYGNSFAYWGQGTLVTVSSGGGGSGGGGSGGGGSDIVMTQSPDSLAVSLGERATINCKSSQSLLNSGNQKNYLTWYQQKPGQPPKLLIYWASTRESGVPDRFSGSGSGTDFTLTISSLQAEDVAVYYCQNDYSYPLTFGGGTKVEIKR

SEQ ID NO: 30 - protein, 247 aa, Artificial Sequence
  Description of artificial sequence artificially synthesized sequence
QVQLQESGPGLVKPSQTLSLTCTVSGGSISSGYNWHWIRQPPGKGLEWIGYIHYTGSTNYNPALRSRVTISVDTSKNQFSLKLSSVTAADTAVYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 31 - protein, 247 aa, Artificial Sequence
  Description of artificial sequence artificially synthesized sequence
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELKR

SEQ ID NO: 32 - protein, 247 aa, Artificial Sequence
  Description of artificial sequence artificially synthesized sequence
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKR

SEQ ID NO: 33 - protein, 246 aa, Artificial Sequence
  Description of artificial sequence artificially synthesized sequence
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 34 - protein, 226 aa, Artificial Sequence
  Description of artificial sequence artificially synthesized sequence
TTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 35 - protein, 224 aa, Artificial Sequence
  Description of artificial sequence artificially synthesized sequence
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - protein, 268 aa, Artificial Sequence
  Description of artificial sequence artificially synthesized sequence
TTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 37 - DNA, 1419 bp, Artificial Sequence
  Description of artificial sequence artificially synthesized sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 38 - DNA, 1413 bp, Artificial Sequence
  Description of artificial sequence artificially synthesized sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 39 - DNA, 1545 bp, Artificial Sequence
  Description of artificial sequence artificially synthesized sequence
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggccaacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

(19) United States
(12) Patent Application Publication
(10) Pub. No.: US 2024/0285761 A1
(43) Pub. Date: Aug. 29, 2024

(54) METHOD AND COMPOSITION FOR TREATING TUMORS

(71) Applicant: CARSGEN THERAPEUTICS CO., LTD., Shanghai (CN)

(72) Inventors: Zonghai LI, Shanghai (CN); Wei WANG, Shanghai (CN); Jun XIAO, Shanghai (CN); Daijing YUAN, Shanghai (CN)

(73) Assignee: CARSGEN THERAPEUTICS CO., LTD., Shanghai (CN)

(21) Appl. No.: 18/573,685

(22) PCT Filed: Jun. 23, 2022
(86) PCT No.: PCT/CN2022/100762

§ 371 Date: Dec. 22, 2023

(30) Foreign Application Priority Data
Jun. 23, 2021 (CN) 202110701218.X
Apr. 8, 2022 (CN) 202210378612.9

(51) Int. Cl. A61K 39/00; A61K 31/337; A61K 31/675; A61K 31/7076; A61K 45/06; A61P 35/00

(52) U.S. Cl. CPC A61K 39/464402; A61K 31/337; A61K 31/675; A61K 31/7076; A61K 39/4611; A61K 39/4631; A61K 45/06; A61P 35/00; A61K 2039/545

(57) ABSTRACT

Provided is a treatment method for administration of immune effector cells to an individual suffering from or suspected of suffering from a CLD18 positive biliary tract tumor, comprising administrating immune effector cells that specifically recognize CLD18.2 to an individual. Pretreatment is performed and then the immune effector cells of the present application are administrated, so that the tumor treatment efficacy of the immune effector cells can be remarkably improved.

### RELATED PATENT APPLICATIONS

This patent application claims priority of Chinese patent application with application number 202110701218.X submitted on Jun. 23, 2021 and the Chinese patent application with application number 20/2210378612.9 submitted on Apr. 8, 2022, the contents of the aforementioned patent applications are incorporated herein by reference in their entirety.

### SEQUENCE LISTING

The instant application contains a Sequence Listing which has been submitted in ASCII format and is hereby incorporated by reference in its entirety. Said ASCII copy, created on Jun. 23, 2022, is named “FF00616PCT-sequence listing.txt”, and is 51.7 kb in size.

### TECHNICAL FIELD

The application belongs to the field of immunotherapy: particularly it relates to immune cell therapy that targets the recognition of tumor antigens, triggers the activation of immune effector cells, and exerts anti-tumor effects.

### BACKGROUND ART

Published clinical research results have showed that, the progression-free survival of biliary tract tumor that has received first-line standard treatment is less than about 6 months. Therefore, there is an urgent need to develop new treatments for biliary tumors or to prolong patient survival.

### CONTENTS OF THE APPLICATION

The application provides a method for treating a subject suffering from or suspected of suffering from CLD18-positive biliary tumor, which comprises administrating cells that express exogenous receptors targeting CLD18 to the subject.

The application also provides use of cells that express exogenous receptors targeting CLD18 in the preparation of a medicament for treating a subject suffering from or suspected of suffering from CLD18 positive biliary tumor.

The application also provides use of cells that express exogenous receptors targeting CLD18 and chemical drugs or other biological drugs or radiotherapy in the preparation of a medicament for treating a subject suffering from or suspected of suffering from CLD18 positive biliary tumor.

The application also provides use of cells that express exogenous receptors targeting CLD18 for treating a subject suffering from or suspected of suffering from CLD18 positive biliary tumor.

The application also provides use of cells that express exogenous receptors targeting CLD18 and chemical drugs or other biological drugs or radiotherapy for treating a subject suffering from or suspected of suffering from CLD18 positive biliary tumors.

In a preferred example, the CLD18 is CLD18.2.

In a preferred example, the biliary tract tumor comprises a gallbladder cancer and a cholangiocarcinoma.

In a preferred example, the cells comprise immune effector cells.

In a preferred example, the immune effector cells are selected from the group consisting of: T cells, NK cells, NKT cells, mastocytes, macrophages, dendritic cells, CIK cells, stem cell-derived immune effector cells, or a combination thereof.

In a preferred example, the immune effector cells are derived from natural T cells and/or T cells induced by pluripotent stem cells.

In a preferred example, the immune effector cells are autologous or allogeneic T cells, or primary T cells.

In a preferred example, the T cells comprise: memory stem cell-like T cells (Tscm cells), central memory T cells (Tcm), effector T cells (Tef), regulatory T cells (Tregs), effector memory T cells (Tem), γδ T cells, αβ T cells, or combinations thereof.

In a preferred example, the exogenous receptor is selected from the group consisting of: chimeric antigen receptor (CAR), T cell receptor (TCR), T cell fusion protein (TFP), T cell antigen coupler (TAC), antibody-TCR chimera or combination thereof; wherein the antigen-binding domain of the exogenous receptor specifically binds to CLD18.2.

In a preferred example, the chimeric antigen receptor comprises:
(i) an antibody specifically binding to CLD18.2, a transmembrane region of CD8 or CD28, and a intracellular domain of CD3ζ;
(ii) an antibody specifically binding to CLD18.2, a transmembrane region of CD8 or CD28, a intracellular domain of CD137, and a intracellular domain of CD3ζ;
(iii) an antibody specifically binding to CLD18.2, a transmembrane region of CD8 or CD28, a intracellular domain of CD28, and a intracellular domain of CD3ζ; or
(iv) an antibody specifically binding to CLD18.2, a transmembrane region of CD8 or CD28, a intracellular domain of CD28, a intracellular domain of CD137, and a intracellular domain of CD3ζ.

In a preferred example, at least one cycle of the cells are administrated to the subject for treatment: preferably, 1-3 cycles of the cells are administrated to the subject for treatment.

In a preferred example, a dose of the cells in a cell therapy product administrated per cycle is no more than about 2×10⁹ cells/kg of subject body weight, about 2×10⁸ cells/kg of subject body weight, or about 2×10⁷ cells/kg of subject body weight: or a dose of the cells in a cell therapy product is no more than about 1×10¹¹ cells/subject, about 1×10¹⁰ cells/subject, about 5×10⁹ cells/subject, about 2×10⁹ cells/subject, or about 1×10⁹ cells/subject.

In a preferred example, a dose of the cells in a cell therapy product administrated per cycle is about 1×10⁵ cells/kg of subject body weight to 2×10⁷ cells/kg of subject body weight, or about 1×10⁶ cells/kg of subject body weight to 2×10⁷ cells/kg of subject body weight: or
a dose of the cells in a cell therapy product administrated per cycle is about 1×10⁷ cells to 5×10⁹ cells/subject, about 1×10⁷ cells/subject to 2×10⁹ cells/subject, or about 1×10⁷ cells/subject to 1×10⁹ cells/subject: or
a dose of the cells in a cell therapy product administrated per cycle is about 1×10⁸ cells/subject to 5×10⁹ cells/subject, about 1×10⁸ cells/subject to 2×10⁹ cells/subject, or about 1×10⁸ cells to 1×10⁹ cells/subject: or
a dose of the cells in a cell therapy product administrated per cycle is about 2.5×10⁸ cells/subject to 5×10⁸ cells/subject.

In a preferred example, a pretreatment is performed before administrating the cell therapy product in each cycle, and the pretreatment comprises administrating chemical drug, biological drug, radiotherapy, or a combination thereof to the subject.

In a preferred example, the pretreatment is performed 1-8 days before administrating the cell therapy product: preferably 2-6 days before administrating the cell therapy product; and preferably each of the chemical drug, the biological drug, the radiotherapy, or a combination thereof is used continuously for no more than 4 days.

In a preferred example, wherein the chemical drug is any one or at least two selected from the group consisting of: cyclophosphamide, fludarabine, tubulin inhibitor, and pyrimidine anti-tumor drug: or the chemical drug comprises cyclophosphamide and fludarabine: or the chemical drug comprises cyclophosphamide, fludarabine, and tubulin inhibitor.

In a preferred example, the tubulin inhibitor comprises taxane compound: or the tubulin inhibitor comprises paclitaxel, albumin-bound paclitaxel, and docetaxel; or the tubulin inhibitor is albumin-bound paclitaxel.

In a preferred example, the dosage of fludarabine is about 10-50 mg/m²/day, or about 15-40 mg/m²/day, or about 15-30 mg/m²/day, or about 20-30 mg/m²/day: or about 25 mg/m²/day, or about 30-60 mg/day, or about 30-50 mg/day, or about 35-45 mg/day; or
the dosage of cyclophosphamide is about 200-400 mg/m²/day, or about 200-300 mg/m²/day, or about 250 mg/m²/day, or about 300-1000 mg/day, or about 300-550 mg/day, or about 300-500 mg/day: or
the dosage of taxane compound is no more than about 300 mg/day, or no more than about 200 mg/day, or about 90-120 mg/day.

In a preferred example, the cyclophosphamide is administrated 2-3 times; or the fludarabine is administrated 1-2 times; or the taxane compound is administrated once.

In a preferred example, the antigen binding domain or the antibody has a scFv sequence represented by SEQ ID NOs: 2, 23, 24, 25, 26, 27, 28, 29, 30, 31, 32 or 33; or
the antigen binding domain or the antibody has a HCDR1 represented by SEQ ID NO: 14, a HCDR2 represented by SEQ ID NO: 15, and a HCDR3 represented by SEQ ID NO: 16, a LCDR1 represented by SEQ ID NO: 17, a LCDR2 represented by SEQ ID NO: 18, and a LCDR3 represented by SEQ ID NO: 19; or
the antigen binding domain or the antibody has a heavy chain variable region represented by SEQ ID NO: 10 and a light chain variable region represented by SEQ ID NO: 12.

In a preferred example, the chimeric antigen receptor has a scFv sequence represented by SEQ ID NOs: 2, 23, 24, 25, 26, 27, 28, 29, 30, 31, 32 or 33 which is sequentially connected to SEQ ID NOs: 34, 35 or 36 respectively; or the chimeric antigen receptor has a nucleotide sequence represented by SEQ ID NOs: 37, 38 or 39.

In a preferred example, before administrating the cell therapy product in each cycle, serum levels of cytokines indicating CRS, cytokines indicating neurotoxicity, indicators indicating tumor burden, and/or factors indicating host anti-CAR immune response for the subject are evaluated.

In a preferred example, after being treated with the cell therapy product, the subject does not show severe CRS, or does not show neurotoxicity exceeding grade 3.

In a preferred example, after the cell therapy product administrated in the prior cycle of treatment are no longer detectable in the body, the treatment of the subsequent cycle is continued.

In a preferred example, the dose of the cells administrated in the subsequent cycle is an amount sufficient to stabilize or reduce the tumor burden of the subject.

In a preferred example, a dose of the cells in each cycle are administrated in 1-5 times, preferably in 1-3 times.

In a preferred example, when administrating the treatment in the subsequent cycle, the subject has any one of the following characteristics:
(i) the peak level of cytokines related to cytokine release syndrome (CRS) in the serum of the subject is smaller than the peak level after administration of the cell therapy product in the prior cycle;
(ii) grade 3 or higher neurotoxicity is not detected;
(iii) the CRS level is reduced as compared with the CRS level after administration of the cell therapy product in the prior cycle;
(iv) the subject does not exhibit a detectable humoral or cell-mediated immune response to the cell therapy product.

In a preferred example, in the above (i), the level of cytokines is reduced by at least 50%, preferably by at least 20%, more preferably by at least 5% as compared with the peak level of cytokines after administration of the cell therapy product in the prior cycle.

In a preferred example, the CRS level is equivalent to the CRS level before administration of the cell therapy product in the prior cycle.

In a preferred example, a pretreatment is performed before administrating the cells in each cycle, and the pretreatment includes administrating chemical drugs and/or radiotherapy to the subject.

In a preferred example, the radiotherapy comprises whole body radiation therapy or local radiation therapy.

In a preferred example, the pretreatment is performed 1-8 days before administration of the cells: preferably the pretreatment is performed 2-6 days before administration of the cells.

In a preferred example, the chemical drug is any one or at least two selected from the group consisting of: cyclophosphamide, fludarabine, tubulin inhibitor, and pyrimidine anti-tumor drug.

In a preferred example, the chemical drug is cyclophosphamide and fludarabine; or cyclophosphamide, fludarabine and a tubulin inhibitor.

In a preferred example, the tubulin inhibitor is a taxane compound:
preferably the taxane compound is selected from the group consisting of: paclitaxel, albumin-bound paclitaxel, and docetaxel;
more preferably the taxane compound is albumin-bound paclitaxel.

In a preferred example, the pyrimidine anti-tumor drug is selected from the group consisting of: 5-fluorouracil, tegadifurum, carmofur, doxifluridine, and capecitabine.

In a preferred example, each chemical drug is used continuously for no more than 4 days.

In a preferred example, the cyclophosphamide is administrated 2-3 times; or the fludarabine is administrated 1-2 times.

In a preferred example, the taxane compound is administrated once.

It should be understood that within the scope of the application, the above-mentioned technical features of the application and the technical features specifically described below (such as examples) can be combined with each other to form new or preferred technical solutions. Due to literature limitations, they will not be described herein one by one.

### BRIEF DESCRIPTION OF THE DRAWINGS

FIG. 1: Cytotoxicity assay of CAR-T cells.

FIG. 2: CAR-T cell killing experiment in vivo, FIGS. 2A and 2B respectively show the measurements of tumor volume and tumor weight.

### DETAILED DESCRIPTION

After extensive and in-depth research, the inventor unexpectedly discovered that a method for treating a subject suffering from biliary tumors (including cholangiocarcinoma and gallbladder cancer) by immune effector cells (for example, T cells) expressing CLDN18.2-CAR and compositions thereof and use thereof. On this basis, the application was completed. In some aspects, the method, immune effector cells and compositions provide or achieve improved or more durable responses or efficacy, and/or reduced risk of toxicity or other side effects as compared with the prior art. In one example, the method significantly improves the anti-biliary tumor efficacy by administrating a specified or relative number of cells to treat a specific subject population.

Published clinical research results show that, the progression-free survival of a subject suffering from biliary tumors that have received first-line standard treatment is less than about 6 months (1.8-5.6 months). In the application, a CAR-T cell therapy is administrated to subjects with gallbladder cancer, which show longer progression-free survival and overall survival, allowing the subjects to obtain significant clinical benefits: after the CAR-T cell therapy, one subject with gallbladder cancer achieved PR (partial response) with a PFS (progression-free survival) of 4.2 months, and OS (Overall Survival, overall survival) of about 10 months; and after the CAR-T cell therapy, another subject with gallbladder cancer achieved SD (stable disease, in a stable state) with a PFS of 9.3 months and continuous stable phase: after the CAR-T cell therapy, one subject with cholangiocarcinoma achieved SD with a PFS of 7.5 months and continuous stable phase.

Unless otherwise defined, all technical terms, symbols and other technical and scientific terms or proprietary words used herein are intended to have the same meanings commonly understood by those skilled in the art. In some cases, terms having conventionally understood meanings are further limited herein for the purpose of clarification and/or ease of reference, and such further limitations comprised herein should not be construed to mean that they are substantially different from those commonly understood in the art.

All publications involved in the application, including patent documents, academic papers, and databases, are independently incorporated by reference in their entirety. To the extent that definitions set forth herein differ or are otherwise inconsistent with definitions set forth in patents, published applications, and other publications incorporated herein by reference, the definitions set forth herein shall control.

### I. Definition

As used herein, “about” may mean depending on the specific circumstances and known or knowable by those skilled in the art, or may mean changing in a range up to about ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±25%, or ±30% based on a given value. That is to say, the range modified by “about” covers a range of: the given value ±1%, the given value ±2%, the given value ±3%, the given value ±4%, the given value ±5%, the given value ±6%, the given value ±7%, the given value ±8%, the given value ±9%, the given value ±10%, the given value ±11%, the given value ±12%, the given value ±13%, the given value ±14%, the given value ±15%, the given value ±16%, the given value ±17%, the given value ±18%, the given value ±19%, the given value ±20%, the given value ±25%, the given value ±30%. Alternatively, specifically with respect to biological systems or methods, the term may refer to being within an order of magnitude of a value, such as within about 5 times or within about 2 times a value.

Scope: descriptions in range form are merely for convenience and brevity, and should not be construed as an immutable limitation on the scope of the application. Accordingly, descriptions of ranges should be considered as specifically disclosing all possible subranges as well as individual values within the range.

When describing an amino acid or nucleic acid sequence, “having” a particular sequence in the application should be understood to encompass variants of the specific sequence. The amino acid or nucleic acid sequence in the application has a certain specific sequence, means that the amino acid or nucleic acid sequence has more than 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the specific sequence. Sequence identity can be measured by sequence analysis softwares (programs such as BLAST, BESTFIT, GAP, PILEUP/PRETTYBOX, etc.). Such software matches identical or similar sequences by assigning degrees of homology to various substitutions, deletions, and/or other modifications. Conservative substitutions generally comprise substitutions within the following groups: glycine, and alanine; valine, isoleucine, and leucine: aspartic acid, glutamic acid, asparagine, and glutamine; serine, threonine acid, lysine, and arginine; and phenylalanine, and tyrosine. In an exemplary method for determining the degree of identity, the BLAST program can be used, wherein a probability score between e-3 and e-100 indicates closely related sequences.

The term “exogenous” refers to a nucleic acid molecule or polypeptide that is not endogenously present in a cell, or its expression level is insufficient to achieve the function when it is overexpressed: the term covers any recombinant nucleic acid molecule or polypeptide expressed in a cell, such as exogenous, heterologous and overexpressed nucleic acid molecule or polypeptide. In one example, an exogenous receptor comprises a chimeric receptor, which refers to a fusion molecule formed by connecting DNA fragments from different sources, or corresponding cDNA or polypeptide fragments of proteins by using genetic recombination technology, including extracellular domains, transmembrane domains and intracellular domains. Exogenous receptors include, but are not limited to, chimeric antigen receptor (CAR), chimeric T cell receptor (TCR), T cell antigen coupler (TAC), and T cell fusion protein (TFP).

The term “CAR” comprises extracellular antigen-binding domain, transmembrane domain, and intracellular signaling domain. In one example, the extracellular antigen binding domain of the CAR comprises scFv. The intracellular signaling domain comprises the functional signaling domain of the stimulatory molecule and/or the costimulatory molecule; or the entire intracellular portion of the stimulatory molecule and/or the costimulatory molecule, or the entire native intracellular signaling domain, or a functional fragment or derivative thereof.

The term “T cell receptor (TCR)” mediates T cell recognition of specific major histocompatibility complex (MHC)-restricted peptide antigens, and includes native TCR receptors and modified TCR receptors. Natural TCR receptors are composed of two peptide chains, α and β. Each peptide chain can be divided into variable region (V region), constant region (C region), transmembrane region and cytoplasmic region etc., with antigen specificity exists in the V region. After modification of the natural TCR, a modified TCR receptor that can specifically bind to CLD18.2 polypeptide is obtained. Modified TCR receptors herein include, but are not limited to, chimeric T cell receptors (TCR), T cell antigen couplers (TAC), T cell fusion proteins (TFP), and antibody-TCR chimeras.

“Specific binding” means that a polypeptide or fragment thereof recognizes and binds to a biological molecule of interest (e.g., a polypeptide), but does not substantially recognize and bind to other molecules in a sample. Binding affinity can be determined by using standard binding assays.

The term “antigenic domain” refers to molecules that specifically bind to an antigenic determinant, including immunoglobulin molecules and immunologically active portions of immune molecules, i.e., molecules that contain an antigen-binding site that specifically binds to an antigen (an “immune response”). The term “antibody” comprises not only whole antibody molecules, but also fragments of antibody molecules that retain antigen-binding ability. The term “antibody” is used interchangeably with the terms “immunoglobulin” and “antigenic domain” in the application. Antibodies include, but are not limited to: monoclonal antibodies, polyclonal antibodies, natural antibodies, bispecific antibodies, chimeric antibodies, Fv, Fab, Fab′, Fab′-SH, F(ab′)2, linear antibodies, single chain antibody molecules (e.g. scFv), single domain antibodies. In one example, the antibody comprises at least two heavy (H) chains and two light (L) chains linked by disulfide bonds. Each heavy chain is composed of a heavy chain variable region (VH) and a heavy chain constant region (CH). The CH is composed of three domains: CH1, CH2, and CH3. Each light chain is composed of a light chain variable region (VL) and a light chain constant region (CL). The CL is composed of a structural domain. VH and VL can be further subdivided into hypervariable regions called complementarity-determining regions (CDRs), interspersed with more conservative regions called framework regions (FRs). Each VH and VL is composed of three CDRs and four FRs, arranged in the following order from amino terminus to carboxyl terminus: FR1, CDR1, FR2, CDR2, FR3, CDR3, and FR4. The variable regions of the heavy and light chains comprise binding domains that interact with the antigen. The constant regions of antibodies mediate the binding of immunoglobulins to host tissues or factors, comprising various cells of the immune system (e.g., immune effector cells) and the first component (Clq) of the classical complement system. An antigenic domain “specifically binds” or is “immunoreactive” to an antigen, if the antigenic domain binds to the antigen with greater affinity than it binds to other reference antigens (including polypeptides or other substances).

The term “composition” refers to a mixture of two or more substances. The composition can be a solution, suspension, liquid, powder, paste, aqueous, non-aqueous or any combination thereof.

The terms “activation” and “activating” are used interchangeably, and can refer to a process by which cells transition from a quiescent to an active state. The process may comprise a response to phenotypic or genetic change in antigen, migration and/or functional activity states.

The term “nucleic acid” or “polynucleotide” refers to deoxyribonucleic acid (DNA) or ribonucleic acid (RNA) and their polymers in single- or double-stranded form, comprising any nucleic acid molecule encoding a polypeptide of interest or a fragment thereof. The nucleic acid molecule only needs to maintain substantially identity with the endogenous nucleic acid sequence, and does not need to be 100% homologous, or identical with the endogenous nucleic acid sequence. A polynucleotide “substantially identical” to an endogenous sequence is generally capable of hybridizing to at least one strand of a double-stranded nucleic acid molecule. The term “substantial identity” or “substantial homology” refers to a polypeptide or nucleic acid molecule exhibiting at least about 50% homology or identity with a reference amino acid sequence or nucleic acid sequence.

The terms “peptide,” “polypeptide,” and “protein” are used interchangeably, and refer to compounds consisting of amino acid residues covalently linked by peptide bonds.

### II. Exogenous Receptor

The exogenous receptor in the application refers to a fusion molecule formed by connecting DNA fragments from different sources or cDNA corresponding to proteins by genetic recombination technology, comprising the extracellular domain, transmembrane domain and intracellular domain that specifically bind to CLD18, including but not limited to: chimeric antigen receptor (CAR), chimeric T cell receptor (TCR), T cell antigen coupler (TAC), T cell fusion protein (TFP), antibody-TCR chimera. In one example, the application provides an exogenous receptor that specifically binds to CLD18.2 polypeptide. In one example, the exogenous receptor binds to the extracellular domain of the CLD18.2 polypeptide. In one example, the application provides a CAR that specifically binds to a CLD18.2 polypeptide.

CLD18 polypeptide (Claudin 18, CLDN18) refers to CLD18 gene or any variant, derivative or isoform of the encoded protein, comprising splice variant 1 (Claudin 18.1, CLD18.1, CLDN18.1): NP_057453, NM016369, and splice variant 2 (Claudin 18.2, CLD18.2, CLDN18.2): NP_001002026, NM_001002026. In one example, the CLD18.2 polypeptide is a human CLD18.2 polypeptide, comprising an amino acid sequence or a fragment thereof having at least about 80%, 85%, 90%, 95%, 96%, 97%, 98%, 99% or 100% homology or identity with the sequence represented by SEQ ID NO:9, and/or may optionally comprise at most one or at most two or at most three conservative amino acid substitutions. CLD18.2 polypeptide is strongly expressed in several cancer types, including gallbladder cancer and cholangiocarcinoma.

### 2.1 CAR

In one example, the exogenous receptor encompassed by the application is a CAR that specifically binds to a CLD18 polypeptide. In one example, the CAR specifically binds to a CLD18.2 polypeptide. In one example, the CAR specifically binds to the extracellular domain of a CLD18.2 polypeptide. In one example, the various domains of the CAR polypeptide are in the same polypeptide chain, e.g., expressed as a single polypeptide chain. In some examples, the various domains of a CAR polypeptide are not contiguous with each other, e.g., are in different polypeptide chains.

### 2.1.1 Antigenic Domain

In one example, the antigenic domain comprises an antibody. In one example, the antigenic domain comprises scFV. In one example, the antigenic domain comprises a heavy chain variable region (VH) and/or a light chain variable region (VL) of an antibody: or comprises a cross-linked Fab; or comprises F(ab)₂. In one example, the antigenic domain comprises antibody VH and VL, forming a variable fragment (Fv).

In one example, the CAR comprises an antibody specifically binding to a CLD18.2 polypeptide. In one example, the antigenic domain of the CAR comprises an Fv that specifically binds a CLD18.2 polypeptide. In one example, the antigen-binding domain of the CAR comprises: HCDR1 represented by SEQ ID NO: 14, HCDR2 represented by SEQ ID NO: 15, HCDR3 represented by SEQ ID NO: 16, and LCDR1 represented by SEQ ID NO: 17, LCDR2 represented by SEQ ID NO: 18, LCDR3 represented by SEQ ID NO: 19. In one example, the antigen-binding domain of the CAR comprises: VH represented by SEQ ID NO: 10, VL represented by SEQ ID NO: 12. In one example, the antigen-binding domain of the CAR comprises: the scFv sequence represented by SEQ ID NOs: 2, 23, 24, 25, 26, 27, 28, 29, 30, 31, 32 or 33.

In one aspect, the application contemplates modification of the amino acid sequence of a starting antibody (e.g., VH or VL) that results in a functionally equivalent molecule. For example, the modified CAR comprises a sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity with the sequence specifically binds to the VH or VL of the antigenic domain of CLD18.2 peptide.

In one example, the antibodies of the application can be further modified such that they vary in amino acid sequence (e.g., relative to wild type) but not in the desired activity. For example, additional nucleotide substitutions can be applied to the protein, resulting in amino acid substitutions at “non-essential” amino acid residues. For example, a non-essential amino acid residue in a molecule can be substituted by another amino acid residue from the same side chain family. In another embodiment, an amino acid fragment may be substituted by an amino acid fragment that are structurally similar but differ in sequence and/or composition from the side chain family members, e.g., conservative substitutions may be performed in which the an amino acid residue is substituted by an amino acids with similar side chain.

In one example, the antigenic domain is bound to the transmembrane domain directly or through a hinge. In one example, the hinge comprises a CD8 hinge, e.g., a sequence having 95-100% identity with the sequence represented by SEQ ID NO:3.

In one example, the upstream of a CAR-encoding nucleic acid molecule comprises a polynucleotide encoding a signal peptide. In one embodiment, the sequence of a signal peptide has the sequence represented by SEQ ID NO:1.

### 2.1.2 Transmembrane Domain

The CAR can be anchored to the cell membrane by a transmembrane domain. The transmembrane domain of the CAR molecule of the application comprises a transmembrane domain selected from the group consisting of: the α, —B, or ζ transmembrane region of the T cell receptor, the transmembrane domain of CD28, CD38, CD45, CD4, CD5, CD8, CD9, CD16, CD22, CD33, CD37, CD64, CD80, CD86, CD134, CD154, KIRDS2, OX40, CD2, CD27, LFA-1(CD11a, CD18), ICOS(CD278), 4-1BB(CD137), GITR, CD40, BAFFR, HVEM(LIGHTR), SLAMF7, NKp80(KLRF1), CD160, CD19, IL2Rβ, IL2Rγ, IL7Rα, ITGA1, VLA1, CD49a, ITGA4, IA4, CD49D, ITGA6, VLA-6, CD49f, ITGAD, CDIld, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4(CD244, 2B4), CD84, CD96(Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D and/or NKG2C.

In one example, the CAR comprises a CD8 transmembrane region, a sequence having 95-100% identity with the sequence represented by SEQ ID NO:4. In one example, the CAR comprises a CD28 transmembrane region, a sequence having 90-100% identity with the sequence represented by SEQ ID NO:5.

### 2.1.3 Intracellular Signaling Domain

In one example, the CAR provided herein comprises an intracellular signaling domain. In one example, the intracellular signaling domain comprises a primary signaling domain. In one example, the intracellular signaling domain comprises a costimulatory signaling domain. In one example, the intracellular signaling domain comprises a primary signaling domain and a costimulatory signaling domain.

In one example, the primary signaling domain comprises a functional signaling domain of a protein selected from the group consisting of: CD3ζ, CD3γ, CD3δ, CD3ε, FcRγ (FCER1G), FcRβ (FcεR1b), CD79a, CD79b, FcγRIIa, DAP10, and DAP12. In one example, the primary signaling domain comprises an intracellular domain of CD3ζ, a sequence having 90-100% identity with the sequence represented by SEQ ID NO:8.

In one example, the costimulatory signaling domain comprises functional signaling domain(s) of one or more proteins selected from the group consisting of: CD27, CD28, CD137, OX40, CD30, CD40, PD-1, ICOS, lymphocyte function-related antigen-1(LFA-1), CD2, CD7, LIGHT, NKG2C, B7-H3, a ligand that specifically binds to CD83, CDS, ICAM-1, GITR, BAFFR, HVEM(LIGHTR), SLAMF7, NKp80(KLRF1), CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1. ITGAM, CD11b, ITGAX, CD11c, ITGB1, CD29, ITGB2, CD18, LFA-1, ITGB7, TNFR2, TRANCE/RANKL, DNAM1(CD226), SLAMF4(CD244,2B4), CD84, CD96(Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, or NKG2D.

In one example, the costimulatory signal domain comprises a CD137 intracellular domain, a sequence having 90-100% identity with the sequence represented by SEQ ID NO:6.

In one example, the intracellular signaling domain of the CAR comprises a human CD3ζ intracellular domain. In one example, the intracellular signaling domain of the CAR comprises a human CD3ζ intracellular domain and a CD28 intracellular domain. In one example, the intracellular signaling domain of the CAR comprises a human CD3ζ intracellular domain and a CD137 intracellular domain. In one example, the intracellular signaling domain of the CAR comprises a CD3ζ intracellular domain, a CD28 intracellular domain, and a CD 137 intracellular domain.

The application contemplates modification of the entire CAR molecule, for example, the modification of one or more amino acid sequences of various domains of the CAR molecule, so as to produce a functionally equivalent molecule. The modified CAR molecule maintains at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with the starting CAR molecule.

Exemplary exogenous receptors, such as sequences comprising CAR have the scFv sequence represented by SEQ ID NOs: 2, 23, 24, 25, 26, 27, 28, 29, 30, 31, 32 or 33 which is connected sequentially to the sequence represented by SEQ ID NOs: 34, 35 or 36, respectively. For example, the sequence represented by SEQ ID NO: 2 is connected sequentially to the sequence represented by SEQ ID NO: 34, or the sequence represented by SEQ ID NO: 2 is connected sequentially to the sequence represented by SEQ ID NO: 35, the sequence represented by SEQ ID NO: 2 is connected sequentially to the sequence represented by SEQ ID NO: 36: the sequence represented by SEQ ID NO: 23 is connected sequentially to the sequence represented by SEQ ID NO: 34, or the sequence represented by SEQ ID NO: 23 is connected sequentially to the sequence represented by SEQ ID NO: 35, the sequence represented by SEQ ID NO: 23 is connected sequentially to the sequence represented by SEQ ID NO: 36: the sequence represented by SEQ ID NO: 24 is connected sequentially to the sequence represented by SEQ ID NO: 34, or the sequence represented by SEQ ID NO: 24 is connected sequentially to the sequence represented by SEQ ID NO: 35, the sequence represented by SEQ ID NO: 24 is connected sequentially to the sequence represented by SEQ ID NO: 36: the sequence represented by SEQ ID NO: 25 is connected sequentially to the sequence represented by SEQ ID NO: 34, or the sequence represented by SEQ ID NO: 25 is connected sequentially to the sequence represented by SEQ ID NO: 35, or the sequence represented by SEQ ID NO: 25 is connected sequentially to the sequence represented by SEQ ID NO: 35, the sequence represented by SEQ ID NO: 25 is connected sequentially to the sequence represented by SEQ ID NO: 36: the sequence represented by SEQ ID NO: 26 is connected sequentially to the sequence represented by SEQ ID NO: 34, or the sequence represented by SEQ ID NO: 26 is connected sequentially to the sequence represented by SEQ ID NO: 35, the sequence represented by SEQ ID NO: 26 is connected sequentially to the sequence represented by SEQ ID NO: 36: the sequence represented by SEQ ID NO: 27 is connected sequentially to the sequence represented by SEQ ID NO: 34, or the sequence represented by SEQ ID NO: 27 is connected sequentially to the sequence represented by SEQ ID NO: 35, the sequence represented by SEQ ID NO: 27 is connected sequentially to the sequence represented by SEQ ID NO: 36; the sequence represented by SEQ ID NO: 28 is connected sequentially to the sequence represented by SEQ ID NO: 34, or the sequence represented by SEQ ID NO: 28 is connected sequentially to the sequence represented by SEQ ID NO: 35, the sequence represented by SEQ ID NO: 28 is connected sequentially to the sequence represented by SEQ ID NO: 36: the sequence represented by SEQ ID NO: 29 is connected sequentially to the sequence represented by SEQ ID NO: 34, or the sequence represented by SEQ ID NO: 29 is connected sequentially to the sequence represented by SEQ ID NO: 35, the sequence represented by SEQ ID NO: 29 is connected sequentially to the sequence represented by SEQ ID NO: 36: the sequence represented by SEQ ID NO: 30 is connected sequentially to the sequence represented by SEQ ID NO: 34, or the sequence represented by SEQ ID NO: 30 is connected sequentially to the sequence represented by SEQ ID NO: 35, the sequence represented by SEQ ID NO: 30 is connected sequentially to the sequence represented by SEQ ID NO: 36; the sequence represented by SEQ ID NO: 31 is connected sequentially to the sequence represented by SEQ ID NO: 34, or the sequence represented by SEQ ID NO: 31 is connected sequentially to the sequence represented by SEQ ID NO: 35, the sequence represented by SEQ ID NO: 31 is connected sequentially to the sequence represented by SEQ ID NO: 36: the sequence represented by SEQ ID NO: 32 is connected sequentially to the sequence represented by SEQ ID NO: 34, or the sequence represented by SEQ ID NO: 32 is connected sequentially to the sequence represented by SEQ ID NO: 35, the sequence represented by SEQ ID NO: 32 is connected sequentially to the sequence represented by SEQ ID NO: 36: the sequence represented by SEQ ID NO: 33 is connected sequentially to the sequence represented by SEQ ID NO: 34, or the sequence represented by SEQ ID NO: 33 is connected sequentially to the sequence represented by SEQ ID NO: 35, the sequence represented by SEQ ID NO: 33 is connected sequentially to the sequence represented by SEQ ID NO: 36. Alternatively, the chimeric antigen receptor has the nucleotide sequence represented by SEQ ID NOs: 37, 38 or 39.

### 2.2 Modification of TCR Receptors

The application provides modified TCR receptors that specifically bind to CLD18.2 polypeptides, including but not limited to: chimeric T cell receptors, T cell fusion proteins (TFP), T cell antigen couplers (TAC), and antibody-TCR chimeras. In one example, the antigen-binding domain of the modified TCR receptor has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with the sequence represented by SEQ ID NOs: 2, 23, 24, 25, 26, 27, 28, 29, 30, 31, 32 or 33.

### 3. Host Cells

Host cells provided herein are cells that express exogenous receptors specifically binding to CLD18.2 polypeptide. Exogenous receptors can activate the cells after binding to the CLD18.2 polypeptide. In one example, host cells comprise stem cells. Stem cells comprise human pluripotent stem cells (including human induced pluripotent stem cells (iPSC) and human embryonic stem cells). In one example, host cells comprise immune effector cells (also known as immune cells). In one example, the host cells are primary cells. The application provides cell therapy products comprising the host cells. The host cells may refer to human or non-human, animal-derived cells.

Immune effector cells of the application comprise cells of the lymphoid lineage. The lymphoid lineage comprising B, T, and natural killer (NK) cells provides antibody production, regulation of the cellular immune system, detection of exogenous reagents in the blood, detection of exogenous cells in a host, etc. Non-limiting examples of the immune effector cells of the lymphoid lineage comprise T cells, natural killer T (NKT) cells and precursors thereof, including embryonic stem cells and pluripotent stem cells (e.g., stem cells that differentiate into lymphoid cells or pluripotent stem cells). T cells comprise lymphocytes that mature in the thymus and are primarily responsible for cell-mediated immunity. T cells participate in the adaptive immune system. T cells comprise any type of T cells, including but not limited to: helper T cells, cytotoxic T cells, memory T cells (including central memory T cells, stem cell-like memory T cells (or stem-like memory T cells), and both effector memory T cells: such as TEM cells and TEMRA cells), regulatory T cells (also called suppressor T cells), natural killer T cells, mucosal-associated invariant T cells, γδ T cells, or αβ T cells. Cytotoxic T cells (CTL or killer T cells) are T lymphocytes capable of inducing the death of infected somatic cells or tumor cells. In one example, the cells of the application are selected from the group consisting of: T cells, NK cells, cytotoxic T cells, NKT cells, macrophages, CIK cells, and stem cell-derived immune effector cells, or a combination thereof. In one example, the immune effector cells are T cells. In one example, T cells comprise CD4+ T cells and/or CD8+ T cells. In one example, immune effector cells comprise CD3+ T cells. In one example, the cells in the composition of the application comprise a cell population collected from PBMC cells after stimulation with CD3 magnetic beads. In one example, the immune effector cells are selected from the group consisting of: T cells, NK cells, NKT cells, mastocyte, macrophages, dendritic cells, CIK cells, stem cell-derived immune effector cells, or a combination thereof. In one example, the immune effector cells are derived from natural T cells and/or T cells induced by pluripotent stem cells. In one example, the immune effector cells are autologous or allogeneic T cells, or primary T cells. In one example, the T cells comprise: memory stem cell-like T cells (Tscm cells), central memory T cells (Tcm), effector T cells (Tef), regulatory T cells (Tregs), effector memory T cells (Tem), γδ T cells, αβ T cells, or a combination thereof.

Cells (eg., T cells) of the application may be autologous, non-autologous (e.g., allogeneic), or derived from engineered progenitor or stem cells in vitro, which can be obtained from many sources, including peripheral blood mononuclear cells (PBMC), bone marrow; lymph node tissue, umbilical cord blood, thymus tissue, tissue from sites of infection, ascites, pleural effusion, spleen tissue, and tumors.

In certain aspects of the application, T cells can be obtained from a blood sample collected from a subject by any number of techniques known to those skilled in the art, such as Ficoll™ isolation technology. In a preferred aspect, cells from the individual's circulating blood are obtained by apheresis. Apheresis products commonly contain lymphocytes, including T cells, monocytes, granulocytes, B cells, other nucleated white blood cells, red blood cells, and platelets. In one aspect, cells collected by apheresis can be washed to remove the plasma fraction, and the cells are placed in an appropriate buffer or culture medium for subsequent processing steps. Multiple rounds of selection may also be used in the context of the application. In some aspects, it is necessary to perform selection procedures and use “unselected” cells during activation and expansion process. “Unselected” cells can also be subjected to other rounds of selection.

The cells of the application are capable of regulating the tumor microenvironment.

The source of unpurified CTL can be any source known in the art, such as bone marrow, fetal, neonatal or adult, or other hematopoietic cell sources, such as fetal liver, peripheral blood or umbilical cord blood. Various techniques can be used to isolate cells. For example, negative selection can initially remove non-CTL, mAbs are particularly useful for identifying markers associated with specific cell lineages and/or differentiation stages of positive and negative selection.

Most of the terminally differentiated cells can initially be removed by relatively crude dissociation. For example, magnetic bead separation can be used initially to remove large numbers of irrelevant cells. In certain embodiments, at least about 80%, commonly at least about 70% of the total hematopoietic cells will be removed prior to isolating the cells.

Procedures for isolation include but are not limited to: density gradient centrifugation: resetting: coupling to particles that alter cell density: magnetic separation with antibody-coated magnetic beads: affinity chromatography; cytotoxicity conjugated to or in combination with mAbs agents, including but not limited to: complement and cytotoxins; and panning by using antibody attached to a solid matrix (e.g., plate, chip, elutriation) or any other convenient technique.

Techniques for separation and analysis include but are not limited to flow cytometry, which can have varying levels of complexity, such as multiple color channels, low-angle and obtuse-angle light scattering detection channels, and impedance channels.

Cells can be selected for dead cells by using dyes associated with dead cells, such as propidium iodide (PI). In certain embodiments, cells are collected in culture medium comprising 2% fetal calf serum (FCS) or 0.2% bovine serum albumin (BSA), or in any other suitable, e.g., sterile isotonic culture medium.

### 4. Vectors

Vectors herein comprise an isolated nucleic acid, and can be used to deliver the isolated nucleic acid into a composition within a cell, including but not limited to: linear polynucleotides, polynucleotides associated with ionic or amphiphilic compounds, plasmids, and viruses. Vectors comprise autonomously replicating plasmids or viruses. Vectors also comprise non-plasmid and non-viral compounds that facilitate the transfer of nucleic acids into cells, such as polylysine compounds, liposomes, and the like.

Genetic modification of the host cells described herein can be accomplished by transducing a substantially homogeneous population of cells with a recombinant nucleic acid molecule. In one example, viral vectors (AAV, retrovirus, or lentivirus) are used to introduce nucleic acid molecules into cells. For example, a polynucleotide encoding an exogenous receptor (e.g., CAR) can be cloned into a retroviral vector. Non-viral vectors can also be used. Any suitable viral vector or non-viral delivery system can be used in the transduction. CARs can be constructed with accessory molecules (e.g., cytokines) in a single polycistronic expression cassette, multiple expression cassettes in a single vector, or multiple vectors. Examples of elements that generate polycistronic expression cassettes include but are not limited to: various viral and non-viral internal ribosome entry sites (IRES, e.g., FGF-1 IRES, FGF-2 IRES, VEGF IRES, IGF-II IRES, NF-κB IRES, RUNXIIRES, p53IRES, hepatitis A IRES, hepatitis C IRES, pestivirus IRES, nonbaculovirus IRES, picornavirus IRES, poliovirus IRES, and encephalomyocarditis virus IRES) and cleavable linkers (for example, 2A peptides, such as P2A, T2A, E2A and F2A peptides).

Other viral vectors that may be used comprise, for example, adenovirus, lentiviral and adeno-associated viral vectors, vaccinia virus, bovine papillomavirus or herpesviruses, such as Epstein-Barr virus.

Non-viral methods can also be used for the genetic modification of immune effector cells. For example, nucleic acid molecules can be introduced into immune effector cells by lipofection, asialoorosomucoid-polylysine conjugation, or microinjection under surgical conditions. Other non-viral gene transfer methods comprise transfection in vitro by using liposomes, calcium phosphate, DEAE dextran, electroporation and protoplast fusion. The nucleic acid molecules can also be first transferred into cell types that can be cultured in vitro (for example, autologous or allogeneic primary cells or their progeny), and then the cells (or their progeny) modified by the nucleic acid molecules can be injected into the subject's target tissue or be injected systemically.

### 5. Administration

The composition of the application comprises the host cells or the cell therapy product. In one example, the composition is provided systemically or directly to a subject to induce and/or enhance an immune response to a CLD18.2 polypeptide, and/or treat, and/or prevent biliary tract tumor. In one example, the composition is injected directly into the organ of interest (e.g., an organ affected by a tumor). Alternatively, the composition is provided to the organ of interest indirectly, such as by administration into the circulatory system (e.g., veins, tumor vasculature). Expansion and differentiation agents can be provided before, simultaneously with, or after administration to increase expansion of T cells, NKT cells, or CTL cells in vitro or in vivo.

The immune effector cells in the composition of the application may comprise purified cell population. Those skilled in the art can readily determine the percentage of immune effector cells of the application in a population by a variety of well-known methods, such as fluorescence-activated cell sorting (FACS). In a population comprising the immune effector cells of the application, suitable ranges for purity are as follows: from about 50% to about 55%, from about 5% to about 60%, and from about 65% to about 70%. In one example, the purity is from about 70% to about 75%, from about 75% to about 80%, or from about 80% to about 85%. In one example, the purity is from about 85% to about 90%, from about 90% to about 95%, or from about 95% to about 100%. Cells can be introduced by injection, catheter, etc.

The composition of the application may be a pharmaceutical composition comprising the immune effector cells of the application or progenitor cells thereof and a pharmaceutically acceptable carrier. Administration mode may be autologous or allogeneic. For example, immune effector cells or progenitor cells can be obtained from one subject, and then administrated to the same subject or to a different compatible subject. Peripheral blood-derived immune effector cells or their progeny (e.g., from in vivo, ex vivo, or ex vivo sources) can be administrated by local injection, including catheter administration, systemic injection, local injection, intravenous injection, or parenteral administration. When administrating the compositions of the application, they may be formulated in unit dose injectable forms (solutions, suspensions, emulsions, etc.).

### 6. Dosage Form

The composition according to the application is provided in the form of sterile liquid preparation, such as isotonic aqueous solution, suspension, emulsion, dispersion, or viscous composition, which may be buffered to a selected pH. Liquid formulations are generally easier to prepare than gels, other viscous compositions, and solid compositions. Additionally, liquid compositions are somewhat more convenient to administrate, especially by injection. On the other hand, viscous compositions may be formulated within an appropriate viscosity range to provide longer contact time with a specific tissue. Liquid or viscous compositions may comprise a carrier, which may be a solvent or dispersion medium including, for example, water, saline, phosphate buffered saline, polyols (e.g., glycerol, propylene glycol, liquid polyethylene glycol, etc.), and a suitable mixture thereof.

Sterile injectable solutions may be prepared by incorporating the immune effector cells of the composition according to the application into a desired amount of an appropriate solvent and incorporating varying amounts of other ingredients as desired. Such compositions may be mixed with suitable carriers, diluents or excipients such as sterile water, physiological saline, glucose, dextrose. The composition may also be freeze-dried. The composition may comprise auxiliary substance such as wetting, dispersing or emulsifying agents (e.g., methylcellulose), pH buffers, gelling or thickening agents, preservatives, flavoring agents, pigments, and the like, which depends on the route of administration and formulation required.

Various additives may be added to enhance the stability and sterility of the composition, including antimicrobial preservatives, antioxidants, chelating agents and buffers. Protection against the action of microorganisms may be ensured by various antibacterial and antifungal agents, such as parabens, chlorobutanol, phenol, sorbic acid, etc. Prolonged absorption of the injectable pharmaceutical forms may be brought about by agents which delay absorption, such as aluminum monostearate and gelatin. However, any media, diluent or additive to be used will have to be compatible with the genetically modified immune effector cells or progenitor cells thereof.

The composition may be isotonic, that is, it may have the same osmotic pressure as blood and/or tears. The desired isotonicity of the composition may be achieved by using sodium chloride or other pharmaceutically acceptable agents, such as glucose, boric acid, sodium tartrate, propylene glycol or other inorganic or organic solutes. Sodium chloride may be particularly useful in buffers containing sodium ions.

If desired, a pharmaceutically acceptable thickening agent may be used to maintain the viscosity of the composition at a selected level. For example, methylcellulose is readily and economically available and easy to use. Other suitable thickeners include, for example, xanthan gum, carboxymethylcellulose, hydroxypropylcellulose, carbomer, and the like. The concentration of the thickening agent may depend on the selected agent. It is important to use an amount sufficient for the desired viscosity. Obviously, the selection of suitable carriers and other additives will depend on the exact route of administration and the property of the particular dosage form, e.g., liquid dosage form (e.g., whether the composition is formulated into a solution, suspension, gel or other liquid form, e.g. time-release form or liquid-filled form).

The number of cells in the composition to be administrated will vary depending on the subject being treated. More effective cells can be administrated in a smaller amount. Precise amount of the effective dose may be determined based on the individual factors for each subject, including his or her size, age, sex, weight, and the subject's condition. Dosages may be readily determined by those skilled in the art from the application and knowledge in the art.

Those skilled in the art may readily determine the amounts of cells and optional additives, mediums and/or carriers in the composition and those administrated in the method. Typically, any additives (other than one or more active cells, and/or one or more reagents) are present in phosphate buffered saline in an amount of 0.001% to 50% (by weight) in the solution, and the active ingredient is present in the order from micrograms to milligrams, for example, from about 0.0001 wt % to about 5 wt %, from about 0.0001 wt % to about 1 wt %, from about 0.0001 wt % to about 0.05 wt %, or from about 0.001 wt % to about 20 wt %, from about 0.01 wt % to about 10 wt % or about 0.05 wt % to about 5 wt %. For any composition to be administrated to an animal or human, the following results may be determined: toxicity, for example by determining the lethal dose (LD) and LD50 in a suitable animal model, for example, rodents such as mice; the dose of the composition, the concentration of the ingredients in the composition, and the time period of the administration of the composition to elicite the appropriate response.

### 7. Treatment Method

The application provides a method for treating a subject suffering from or suspected of suffering from a CLD18 positive biliary tract tumor. The biliary tract tumor includes gallbladder cancer and cholangiocarcinoma. In one example, the method of the application is used to treat a subject whose CLD18.2 polypeptide in the biliary tract tumor cells is “positive”, for example, as detected by immunological detection (such as flow cytometry, immunohistochemistry, etc.), the expression level of CLD18.2 polypeptide in the biliary tract tumor cells is significantly higher than that of isotype-matched controls, and/or is substantially similar to the level of the cells known to be positive for CLD18.2 polypeptide, and/or is significantly higher than the level of the cells known to be negative for CLD18.2 polypeptide. The subject may obtain clinical benefit after being treated with the method according to the application. In one example, the present method is used to treat a subject whose biliary tumor cells are “negative” for CLD18.2 polypeptide, for example, when stained with an antibody specifically binding to CLD18.2 polypeptide, the staining may be immunologically detected at a level that is significantly lower than the level of staining detected by the same procedure under the same other conditions by using isotype-matched controls, and/or is significantly lower than the level of cells known to be positive to CLD18.2 polypeptides, and/or is substantially similar to the level of cells known to be negative for CLD18.2 polypeptide: if the subject can obtain clinical benefit after being treated with the method, the subject is suspected patients with CLD18.2 positive biliary tumors.

Clinical benefit refers to a response or a therapeutic effect of the subject to the method. “Response” used herein refers to: complete response, CR (all target lesions disappear, and the short diameter of all pathological lymph nodes must be reduced to <10 mm), according to the Response Evaluation Criteria in Solid Tumors version 1.1 (RECIST1.1): or partial response, PR: the sum of the diameters of target lesions is reduced by at least 30% as compared with the baseline level: or stable disease (SD): the reduction of target lesions does not reach the PR level, the increase does not reach the PD level, and is somewhere in between, and the minimum value of the sum of diameters may be used as a reference when performing study. Progressive disease (PD) is evaluated, according to the Response Evaluation Criteria for Solid Tumors version 1.1 (RECIST1.1): taking the minimum value of the sum of the diameters of all measured target lesions during the entire experimental study as a reference, the sum of the diameters is relatively increased by at least 20% (the baseline value is used as the reference when the baseline measurement value is the smallest value); in addition, the absolute value of the diameter sum must be increased by at least 5 mm (the appearance of one or more new lesions is also considered as PD). Progression-free survival (PFS) is defined as the time from the first dose of the treatment method of the application to the first appearance of disease progression, or death due to any cause. Overall survival (OS) is measured as the time from the first dose of the treatment method of the application to death due to any cause, and will be analyzed in a manner similar to that described for PFS.

In one example, a response comprises the occurrence of CR or PR after treating with the treatment method of the application. In one example, a response comprises the occurrence of CR, PR or SD after treating with the treatment method of the application: in one example, a response means that a subject who receives the treatment method of the application may obtain obvious clinical benefits, including significantly prolonged PFS and/or OS, such as being prolonged by 2 weeks, 3 weeks, 4 weeks, 1 month, 2 months, 3 months, 4 months, 5 months, 6 months or more.

In one example, after treating with the treatment method of the application, the disease burden is reduced by approximately 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90% or 100%.

In one example, after treating with the method provided in the application, the probability of recurrence is reduced as compared with other methods. For example, in one example, the probability of recurrence or progression after treatment is less than about 80%, less than about 70%, less than about 60%, less than about 50%, less than about 40%, less than about 30%, less than about 20%, less than about 10%, less than about 9%, less than about 8%, less than about 7%, less than about 6%.

“Subject” and “patient” are used interchangeably herein, and refer to an individual treated with cell products, such as a human or other animal, usually a human.

“Treatment” as used herein means an intervention that attempts to modify the course of a disease, to alleviate or reduce, in whole or in part, the disease or the symptoms associated with it. The therapeutic effects include, but are not limited to: preventing the occurrence or recurrence of biliary tract tumor, alleviating symptoms, reducing any direct or indirect pathological consequences of biliary tract tumor, preventing metastasis, slowing down the progression rate of biliary tract tumor, improving or alleviating the status of biliary tract tumor, and reducing or improving prognosis, or delaying the development of biliary tract tumor. The “delaying the development of biliary tract tumor” means delaying, hindering, alleviating, slowing down, stabilizing, suppressing and/or delaying the development of biliary tract tumor. The delay may be of varying time periods, depending on the disease history and/or the individual to be treated. Those skilled in the art should understand that, prevention may be encompassed by sufficient or significant delay (for individuals who has not developed into biliary tumors).

The method according to the application comprises administrating the host cells, cell therapy product or composition of the application to a subject. In one example, a subject is administrated an effective amount or a therapeutically effective amount of the host cells, cell therapy product or composition of the application.

As used herein, an “effective amount” or a “therapeutically effective amount” refers to a dose sufficient to prevent or treat biliary tumors in an individual. Effective doses for therapeutic or prophylactic use depend on the stage and severity of the biliary tract tumor being treated, the age, weight and general health of the subject, and the judgment of the prescribing physician. The amount of the dose will also depend on the selected active substance, the method of administration, the time period and frequency of administration, the presence, property and extent of adverse side effects that may accompany the administration of the particular active substance, and the desired physiological effect. One cycle or multiple cycles of administration of the host cells, cell therapy products or compositions described herein may be required, based on the judgment of the prescribing physician or those skilled in the art.

“Tumor burden” used herein comprises the size or degree of differentiation of the tumor, or the type and stage of metastasis, and/or the appearance and disappearance of common complications of intermediate and advanced cancer, such as cancerous pleural and ascites, and/or the appearance of tumor markers and the change of the expression level thereof, and/or the likelihood or incidence of toxic outcomes in a subject, such as CRS, macrophage activation syndrome, tumor lysis syndrome, neurotoxicity and/or immune response of host cells to the administrated cells and/or chimeric antigen receptors. In one example, the size of a tumor is measured by using the rulers that come with PET (positron emission computed tomography) and CT (computed tomography). Tumor markers refer to a type of substances that are characteristically present in malignant tumor cells, or are abnormally produced by malignant tumor cells, or are substances produced by stimulating response of the host to the tumor, and they can reflect the occurrence and development of the tumor and monitor the tumor's response to treatment. Tumor markers exist in the tissues, body fluids and excreta of a tumor patient, and may be detected by immunological, biological and chemical methods. Examples of the tumor markers include alpha-fetoprotein (AFP), CA125, CA15-3, squamous Cellular cancer antigen (SCC), soluble fragment of cytokeratin 19 (CYFRA21-1), carcinoembryonic antigen (CEA), CA199, CA724, etc.

“Cycle” used herein refers to a time period from the baseline period (i.e., the evaluation phase) before receiving treatment of each cell therapy to next the baseline period before the next treatment: for example, from the beginning of the administration of pretreatment drugs to the beginning of next cycle before the administration of pretreatment drugs. In some embodiments, pretreatment is required before administration of the cell therapy product in each cycle.

“Pretreatment” used herein refers to administrating other drugs or treatments to a subject before administrating the host cells, cell therapy products or compositions to the subject, so that the subject's physical condition is more suitable for infusion of the host cells, cell therapy products or compositions. In one example, pretreatment includes administrating chemical drugs, biological drugs, radiation therapy, or a combination thereof to a subject. Preferably chemical drugs are administrated for pretreatment, more preferably the chemical drugs are chemotherapeutic drugs. In one example, the lymphocytes in a subject are reduced by about 50%, 55%, 60%, 65%, or 70% after receiving pretreatment than before pretreatment.

“Chemical drugs” used herein refers to anti-cancer drugs prepared by chemical synthesis methods, including traditional chemotherapy drugs, such as alkylating agents, anti-metabolites, anti-cancer antibiotics, etc., and also including targeted drugs, such as afatinib, alectinib, etc. In one example, chemical drugs include, but are not limited to: alkylating agents, antimetabolites, anti-tumor antibiotics, plant-based anti-cancer drugs, hormones, immune preparations, etc.: for example, diterpene alkaloids (such as taxanes), cyclophosphamide, fludarabine, cyclosporine, rapamycin, melphalan, bendamustine, asparaginase, busulfan, carboplatin, cisplatin, daunorubicin doxorubicin, fluorouracil, hydroxyurea, methotrexate, rituximab, vinblastine and/or vincristine, etc. In one example, the antimetabolites include, but are not limited to: carmofur, tegafur, pentostatin, doxifluridine, trimetreate, fludarabine, gimeracil, oteracil potassium, tegadifur, carmofur, capecitabine, galocitabine, cytarabine sodium octadecylphosphate, fosteabine sodium hydrate, raltitrexed, paltitrexid, emitefur, thiazofurin, nolatrexed, pemetrexed, nelzarabine, 2′-deoxy-2′-methylenecytidine, 2′-fluoromethylene-2′-deoxycytidine, N-[5-(2,3-dihydro-benzofuranyl)sulfonyl]-N′-(3,4-dichlorophenyl)urea, N6-[4-deoxy-4-[N2-[2(E),4(E)-tetradecadienoyl]glycylamino]-L-glycerol-B-L-manno se-pyranoheptanosyl]adenine, aplidine, ecteinascidin, 4-[2-amino-4-oxo-4,6,7,8-tetrahydro-3H-pyrimido[5,4-b]thiazin-6-yl-(S)-ethyl]-2,5-th ienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradec-2,4,6-trien-9-yl acetate, swainsonine, lometrexol, dexrazoxane, methioninase, 2′-cyano-2′-deoxy-N4-palmitoyl-1-B-D-arabinofuranosyl cytosine, and 3-aminopyridine-2-aldehyde thiosemicarbazone, etc. In one example, the alkylating agents include, but are not limited to: dacarbazine, melphalan, cyclophosphamide, temozolomide, chlorambucil, busulfan, nitrogen mustard, nitrosourea, and the like. “Biological drugs (i.e., other biological drugs)” as used herein refer to drugs prepared through molecular biology or genetic engineering, such as antibody drugs, ADCs, etc. “Tubulin inhibitors” as used herein comprise tubulin polymerization promoters and tubulin polymerization inhibitors. Tubulin inhibitors include, but are not limited to: taxanes, epothilones, spongiolides, laulimalide, and the like. “Taxanes” as used herein refers to drugs which contain taxane compounds as main ingredients, which have a mother nucleus structure of bridged methylene benzocyclodecene similar to taxanes. In one example, the mother nucleus structure of bridged methylene benzocyclodecene in a taxane compound contains unsaturated bonds, or does not contain unsaturated bonds. In one example, the carbon atoms on the mother nucleus structure of bridged methylene benzocyclodecene in a taxane compound are substituted with heteroatoms selected from the group consisting of: N, O, S, and P. In one example, the taxane compound is administrated by injection.

In one example, the pretreatment includes administrating to the subject any one or at least two of: cyclophosphamide, fludarabine, tubulin inhibitors and pyrimidine anti-tumor drugs, or a combination thereof.

In one example, before administrating the host cells, cell therapy product or composition in each cycle, the subject is pre-treated, and the pretreatment includes administrating chemotherapy drugs, biological drugs and/or radiotherapy to the subject. In one example, the radiation therapy is whole body radiation therapy or local radiation therapy.

As used herein, “dose” is a dose calculated on a weight basis, or a dose calculated on a body surface area (BSA) basis, or a dose calculated on an individual person basis. The dose calculated based on weight is a dose calculated based on the body weight of a subject, such as mg/kg body weight of a subject or patient, number of cells/kg body weight of a subject or patient, etc. The dose calculated based on BSA is a dose calculated based on the surface area of the subject, such as mg/m², cell number/m², etc. The dose calculated on an individual basis refers to a dose administrated to each subject per cycle or each time, such as mg/subject, number of cells/subject.

“Number of administrations” as used herein refers to the frequency with which the host cells, cell therapy product or composition, or each pretreatment drug is administrated per cycle. For example, the frequency of administration of fludarabine: once: or once a day for 4, 3 or 2 consecutive days. Frequency of administration of cyclophosphamide: once: or once a day for 4, 3 or 2 consecutive days. Frequency of administration of albumin-bound paclitaxel: once: or once a day for 4, 3 or 2 consecutive days. For example, the host cells, cell therapy product or composition administrated in each cycle are administrated once; or administrated in 2, 3, 4, 5, 6, 7, 8, 9, or 10 times, and may be administrated on consecutive days, or be administrated at an interval of 1, 2, 3, 4, 5, or 6 days.

In one example, the subject is administrated at least one cycle of the host cells, cell therapy product or composition for treatment. In one example, the dosage for each cycle may be the same or different. In one example, the dose within a cycle is administrated to a subject once or multiple times, and the divided doses for each administration may be the same or different. For example, the host cells, cell therapy product or composition in one cycle are administrated to the subject once, or divided into 2, 3, 4, 5, or more times. In one example, when the dosage within one cycle is administrated to the subject multiple times, the dosage of the host cells, cell therapy product or composition or pretreatment drug administrated each time is determined by the doctor according to the specific conditions of the subject. The specific conditions of the subject may comprise the subject's overall health, the severity of the disease, the response to the previous dose of the same cycle, the response to the prior cycle, the subject's combined medication, the degree or possibility of toxic reactions, comorbidities, cancer metastasis, and any other factors deemed by the physician to affect the dose of cell therapy or pre-treatment drugs that the subject receives. In one example, the dose is administrated in an increasing manner for each time. In one example, the dose is administrated in a decreasing manner for each time. In one example, the dose is administrated in an increasing manner first and then a decreasing manner for each time. In one example, the dose is administrated in a decreasing manner first, and then in an increasing manner for each time.

Administration of the host cells, cell therapy product or composition for multiple cycles refers to administrating a certain total amount of the host cells, cell therapy product or composition in each time period for multiple time periods. In one example, the intervals between any two adjacent time periods are consistent. In one example, the intervals between any two adjacent time periods are inconsistent. In one example, the host cells, cell therapy product or composition are administrated for at least 2 cycles, such as 2, 3, 4, 5, 6, 7, 8, 9 or 10 cycles. In one example, the design of the dosage and number of administrations per cycle, and the intervals between different cycles, are evaluated with the goal of improving one or more outcomes. For example, the outcome may be a reduction of the extent or likelihood of side effects, or may be an improvement of the efficiency.

In one example, the dose of cells (e.g., CLD18.2-CAR-T cells) that express an exogenous receptor specifically binding to CLD18.2 polypeptide is administrated per cycle at a dose of: no more than about 2×10⁹ cells/kg of subject body weight, or no more than approximately 1×10¹¹ cells/person. Preferably, the dose administrated per cycle is: no more than about 2×10⁸ cells/kg of subject body weight, or no more than about 1×10¹⁰ cells/person. More preferably, the dose administrated per cycle is: no more than about 2×10⁷ cells/kg of subject body weight, or is no more than about 5×10⁹ cells/person, or 2×10⁹ cells/person, or 1×10⁹ cells/person, or 5×10⁸ cells/person. In one example, the dose administrated per cycle is: from about 1×10⁵ cells/kg to 2×10⁷ cells/kg of subject body weight, or from about 1×10⁶ cells/kg to 2×10⁷ cells/kg of subject body weight. In one example, the dose administrated per cycle is: from about 1×10⁷ cells to 1×10⁹ cells, from 1×10⁷ cells to 2×10⁹ cells, from 1×10⁷ cells to 5×10⁹ cells, or from 1×10⁷ cells to 1×10¹⁰ cells: preferably from 1×10⁸ cells to 1×10⁹ cells, from 1×10⁸ cells to 2×10⁹ cells, from 1×10⁸ cells to 5×10⁹ cells; more preferably from 1×10⁸ cells to 5×10⁸ cells, or from 2.5×10⁸ cells to 5×10⁸ cells, from 3.75×10⁸ cells cells to 5×10⁸ cells.

In one example, the pretreatment includes administrating to the subject cyclophosphamide and fludarabine: or cyclophosphamide, fludarabine, and a tubulin inhibitor. In one example, the tubulin inhibitor is a taxane compound. Preferably, the taxane compound is selected from the group consisting of: paclitaxel, albumin-bound paclitaxel, and docetaxel. More preferably, the alkane compound is albumin-bound paclitaxel.

In one example, fludarabine, cyclophosphamide, and albumin-bound paclitaxel may be administrated on the same day, or on different days. If fludarabine, cyclophosphamide, and albumin-bound paclitaxel are administrated on the same day, cyclophosphamide and/or albumin-bound paclitaxel may be administrated before or after fludarabine: or fludarabine and/or albumin-bound paclitaxelb are administrated before or after cyclophosphamide: or cyclophosphamide and/or fludarabine are administrated before or after albumin-bound paclitaxel. In one example, fludarabine, cyclophosphamide, and albumin-bound paclitaxel may be administrated simultaneously or sequentially. In one example, cyclophosphamide is administrated before fludarabine. In one example, cyclophosphamide is administrated after fludarabine. In one example, albumin-bound paclitaxel is administrated before fludarabine. In one example, albumin-bound paclitaxel is administrated after fludarabine. In one example, albumin-bound paclitaxel is administrated before cyclophosphamide. In one example, albumin-bound paclitaxel is administrated after cyclophosphamide.

In one example, fludarabine is administrated in an amount of: about 20-60 mg/day, or about 30-55 mg/day, or about 30-50 mg/day. In one example, fludarabine is administrated in an amount of: about 10-50 mg/m²/day, or about 15-40 mg/m²/day, or about 15-30 mg/m²/day, or about 20-30 mg/m²/day: or about 25 mg/m²/day. In one example, cyclophosphamide is administrated in an amount of: about 200-1000 mg/day, or about 300-600 mg/day, or about 300-560 mg/day, or about 300-550 mg/day, or about 300-500 mg/day. In one example, cyclophosphamide is administrated in an amount of: about 200-400 mg/m²/day, or about 200-300 mg/m²/day, or about 250 mg/m²/day. In one example, the dosage of the taxane compound is no more than about 300 mg/day, or is no more than about 200 mg/day. Preferably, the taxane compound is administrated in an amount of about 90-200 mg/day: more preferably, the taxane compound is administrated in an amount of about 90-120 mg/day. In one example, each pretreatment agent is administrated for no more than 4 consecutive days. In one example, cyclophosphamide is administrated 2-3 times. In one example, fludarabine is administrated 1-2 times. In one example, the taxane is administrated once.

The day that a subject is administrated CAR-T cell therapy for each cycle is designated as day 0. In one example, fludarabine is administrated on day −6 and day −5. In one example, fludarabine is administrated on day −5 and day −4. In one example, fludarabine is administrated on day −4 and day −3. In one example, fludarabine is administrated on day −6, day −5, day −4, and day −3. In one example, fludarabine is administrated on day −7, day −6, day −5, and day −4. In one example, cyclophosphamide is administrated on day −6, day −5, day −4, and day −3. In one example, cyclophosphamide is administrated on day −6, day −5, and day −4. In one example, cyclophosphamide is administrated on day −5, day −4, and day −2. In one example, cyclophosphamide is administrated on day −5, day −4, and day −3. In one example, cyclophosphamide is administrated on day −4, day −3, and day −2. In one example, cyclophosphamide is administrated on day −4 and day −3. In one example, cyclophosphamide is administrated on day −6 and day −5. In one example, cyclophosphamide is administrated on day −5 and day −4. In one example, taxane compound (e.g., albumin-bound paclitaxel) is administrated on day −2, day −3, day −4, day −5, or day −6.

In one example, the use or method described herein further include monitoring the degree or risk of toxic reactions of the subject to the host cells, cell therapy product or composition, and determining subsequent divided dosing or dosage and interval of dosing in subsequent cycles based on the degree or risk of toxicity. In one example, the degree or risk of toxic reactions includes, but is not limited to: CRS, neurotoxicity, macrophage activation syndrome, and tumor lysis syndrome, etc.

In one example, a subsequent cycle treatment is administrated typically after administration of the host cell, cell therapy product or composition, when the risk of a toxic reaction or symptoms thereof or biochemical indicators (such as CRS or neurotoxicity, macrophage activation syndrome or tumor lysis syndrome) is equal to or below acceptable level. In one example, the toxic reaction or symptoms thereof or biochemical indicators comprise one or more of: fever, hypotension, hypoxia, neurological disorders, inflammatory cytokines, and serum levels of C-reactive protein (CRP). In one example, the acceptable level of the risk of a toxic reaction or symptoms thereof or biochemical indicators refers to: 95%, 90%, 85%, 80%, 75%, 70%, 65%, 60%, 55%, 50%, 45%, 40%, 35%, 30%, 25%, 20%, 15%, 10%, or 5% of the peak level, after administration of the prior cycle of treatment. In one example, following treatment of a prior cycle, the treatment of the subsequent cycle is administrated, in the case that the serum level of a factor indicative of cytokine-release syndrome (CRS) in a subject is no more than 5, 6, 7, 8, 9, 10, 11, 12, 13, 14, 15, 16, 17, 18, 19, 20, 21, 22, 23, 24, 25, 26, 27, 28, 29, 30 times as compared to the serum level of the subject prior to administration of the prior cycle. In one example, one or more of subsequent cycle treatments is (are) administrated before an adaptive host immune response (i.e., the body's immune rejection to the CAR-T cells) is generated after the tumor load is stabilized or decreased due to the treatment of the prior cycle. Under such conditions, immune surveillance, clearance, or prevention of proliferation or metastasis of residual tumor cells may be safely and effectively provided in the subsequent-cycles. Thus, in one example, the subsequent-cycle is a disease-consolidating dose.

In one example, the treatment in the subsequent cycle is administrated at least 28, 29, 30, 31, 32, 33, 34, 35 days after administration of the cell therapy product in the prior cycle.

Methods of administrating cell therapy products are known. In one example, the host cells, cell therapy product or composition are administrated to the subject by autologous reinfusion. In one example, the cells that express an exogenous receptor specifically binding to CLD18.2 polypeptide (such as CLD18.2-CAR-T cells) are derived from a subject in need of treatment by the method, and are genetically modified in vitro and then reinfused. In one example, the cells that express an exogenous receptor specifically binding to CLD18.2 polypeptide (e.g., CLD18.2-CAR-T cells) are administrated to the subject by allogeneic reinfusion. In one example, cells that express an exogenous receptor specifically binding to CLD18.2 polypeptide (e.g., CLD18.2-CAR-T cells) are derived from a healthy donor, and the donor and the subject to whom the cells are reinfused are different.

The host cells, cell therapy product or composition may be administrated by any suitable means, for example, by injection, for example, by intravenous or subcutaneous injection, intraperitoneal injection, intraocular injection, fundus injection, subretinal injection, intravitreal injection, counterseptal injection, subscleral injection, intrachoroidal injection, intracameral injection, subconjunctival injection, subconjunctival injection, suprascleral injection, retrobulbar injection, periocular injection, or peribulbar delivery. In one example, the administration is parenteral, intrapulmonary, and intranasal, as well as intralesional. Extraperitoneal infusion includes intramuscular, intravenous, intraarterial, intraperitoneal, or subcutaneous administration. In one example, fludarabine, cyclophosphamide, and albumin-bound paclitaxel may be administrated by any route, including intravenous (IV).

The host cells, cell therapy product or composition (e.g., CAR-T cells) may be administrated by any route, including intravenous injection (IV). In some embodiments, the CAR-T cells are administrated by IV for about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 40 minutes, about 50 minutes, or about 60 minutes.

Various other interventions may be included in the methods described herein. For example, administration of cyclophosphamide with fludarabine may cause adverse events in a subject. The scope of the application includes administrating a composition to a subject such that certain of these adverse events are reduced. In one example, administration of physiological saline to the subject is included. In one example, administration of adjuvants and excipients is included, for example, mesna (sodium 2-mercaptoethane sulfonate). In one example, administration of exogenous cytokines is included.

After administration of the host cells, cell therapy product or composition to a subject, the biological activity of the administrated cell population may be measured by any of a variety of known methods. The parameters for evaluation include: specific binding of cells to antigen, either in vivo (e.g., by imaging) or ex vivo (e.g., by ELISA or flow cytometry). In one example, the biological activity of cells is measured by measuring the expression and/or secretion of cytokines, such as CD10⁷a, IFNγ, IL-2, and TNF. In one example, biological activity measured by evaluation of clinical outcomes, such as reduction in tumor burden. In one example, reduction in tumor markers is evaluated. In one example, cells are evaluated for toxicological consequences, persistence and/or proliferation, and/or the presence or absence of a host immune response.

In one example, if a clinical risk represented by biochemical indexes or indicators (such as cytokine release syndrome (CRS), macrophage activation syndrome, or tumor lysis syndrome, or neurotoxicity) that indicating side effects is absence or eliminated, the subsequent cycle treatment may be administrated if necessary. In one example, whether to administrate a treatment of the subsequent cycle, when to administer a treatment of the subsequent cycle and/or the dosage of a cell therapy product to be administrated in a subsequent cycle is determined based on the presence, absence, or extent of an immune response or a detectable immune response of cells or chimeric antigen receptors expressed by the cells in the prior cycle of the subject. In one example, a subsequent cycle treatment is administrated, when a serum level of a factor indicative of CRS in the subject is no more than about 10 times, 25 times, 50 times, or 100 times of the serum level of the indicator in the subject before administrated the prior cycle. In one example, a subsequent cycle treatment is administrated, when an outcome associated with CRS (e.g., a serum factor associated with or indicative of CRS) or clinical signs or symptoms thereof (such as fever, hypoxia, hypotension, or neurological impairment) has reached peak levels in the subject, and has begun to decline after administrating the prior cycle treatment. In one example, a subsequent cycle treatment is administrated, when the host adaptive immune response has not been detected, has not yet been established, or has not yet reached a certain level, extent or stage. In some aspects, the subsequent cycle treatment is administrated before the development of a memory immune response of a subject.

A detectable immune response refers to an amount detectable by any of the many known methods for evaluating specific immune responses to specific antigens and cells. In one example, by performing ELISPOT, ELISA, or a cell-based antibody detection method (e.g., by flow cytometry) on the subject's serum to detect the presence of antibodies specifically binding to and/or neutralizing the antigen present on the cells, for example, an epitope binding a chimeric antigen receptor (CAR), such as a CAR, to detect a specific type of immune response. In one example, the isotype of the detected antibody is determined, and may indicate the type of response and/or whether the response is a memory response.

In the examples of the application, T cells are prepared from peripheral blood mononuclear cells (PBMC) of human subjects suffering from cancer based on “mononuclear cell apheresis”, and then the T cells are cultured and transduced by viral vectors encoding the chimeric antigen receptor, and the chimeric antigen receptor (CAR) specifically binds to an antigen expressed by tumor cells in the subject, which is a tumor-associated antigen or a tumor-specific antigen. Cells were cryopreserved in infusion medium in individual flexible frozen bags. Each of the bag contains a single unit dose of cells ranging from about 1×10⁶ cells to 5×10⁷ cells. The cells infused per subject in the prior cycle are no more than about 1×10¹² cells, preferably no more than about 1×10¹¹ cells, more preferably no more than about 1×10¹⁰ cells, or about 5×10⁹ cells, or about 2×10⁹ cells. Prior to infusion, the cells are maintained at a temperature of about less than −130° C. or about less than −175° C.

Before starting cell therapy, blood is obtained from the subject and optionally the serum is evaluated for the level of one or more serum factors (such as tumor necrosis factor alpha (TNFα), interferon gamma (IFNγ), IL-10, or IL-6) indicative of cytokine release syndrome (CRS) by methods of ELISA and/or MSD and/or CBA. Tumor burden may optionally be evaluated before the treatment by measuring the size or mass of the solid tumor, for example, by PET or CT scan.

Resuscitation was performed by warming to approximately 38° C., and subject was administrated cells for the prior cycle via multiple infusions. Each infusion is administrated intravenously (IV) as a continuous infusion over approximately 3-30 minutes.

After administration of the prior cycle, the subject undergoes a physical examination and is monitored for any symptoms of toxicity or toxicological outcomes, such as fever, hypotension, hypoxia, neurological deficits, or serum levels of inflammatory cytokines or C-reactive protein (CRP) rise. Optionally, blood is obtained from the subject through one or more times of blood sampling after administration of the prior cycle, and the levels of serum factors indicative of CRS are evaluated by methods of ELISA and/or MSD and/or CBA. Levels of serum factors are compared to the levels of serum factors obtained just before administration of the prior cycle. If necessary, anti-IL6 or other CRS treatment is administrated to reduce symptoms of CRS.

After administration of the prior cycle, for example 1, 2, 3 and/or 4 weeks after the start of administration, optionally the subject is tested for the presence or absence of an anti-CAR immune response, for example, by qPCR, ELISA, ELISPOT, cell-based antibody assays, and/or mixed lymphocyte reactions.

The percentage reduction in tumor burden achieved by a prior cycle may optionally be measured one or more times after the prior cycle in patients suffering from solid tumors by scanning (e.g., PET and CT scans), and/or by quantifying the tumor-positive cells in blood or at tumor site.

The subjects are periodically monitored from the beginning of the first dose, and continuing for up to several years. During follow-up, tumor burden was measured, and/or CAR-expressing cells was detected by flow cytometry and quantitative polymerase chain reaction (qPCR), so as to measure proliferation and persistence of administrated cells in vivo, and/or evaluate anti-CAR development of immune response.

The cells infused in the prior cycle per subject is no more than about 1×10¹² cells, preferably no more than about 1×10¹¹ cells, preferably no more than about 1×10¹⁰ cells, or no more than about 5×10⁹ cells, or no more than 2×10⁹ cells. Prior to the infusion, the cells were maintained at a temperature below −175° C. During the infusion, the temperature is raised to about 38° C. for resuscitation.

The dosage in subsequent cycles is subject-specific, and based on tumor burden, presence of anti-CAR immune response and level of CRS-related outcomes. The dosage in the subsequent cycle is no higher than about 1×10¹² cells, preferably no higher than about 1×10¹¹ cells, more preferably no higher than about 1×10¹⁰ cells, more preferably no higher about 5×10⁹ cells or no higher than about 2×10⁹ cells.

Descriptive statistical methods (such as number of cases, mean, median, standard deviation [SD], minimum and maximum values) were used for analysis. Categorical variables were analyzed by using frequency tables (frequency and percentage). All adverse events (AE) will be classified according to the latest version of the ICH International Dictionary of Medical Terminology MedDRA, and graded according to the Common Standard Terminology Criteria for Adverse Events (CTCAE v5.0 version), and analyzed by using frequency distributions, charts or other descriptive indicators, the number and percentage of subjects experiencing treatment-emergent adverse events (TEAEs) will be calculated by systematic organ classification, preferred terms, and groups. The number of CAR copies in a CAR-T cell including different detection time points will be generated. The Clopper-Pearson method was used to calculate 95% confidence intervals for ORR and DCR.

### 8. Kit

The application provides a kit for treating and/or preventing biliary tumor in a subject. In one example, the kit comprises an effective amount of the host cells, cell therapy product, composition set or pharmaceutical composition according to the application. In one example, the kit comprises a sterile container including a box, an ampoule, a bottle, a vial, a tube, a bag, a sachet, a blister pack, or other suitable container form known in the art. The container may be made of plastic, glass, laminated paper, metal foil, or other materials suitable for containing the drug. In one example, the kit comprises a nucleic acid molecule encoding a CAR of the application, the CAR recognizes a CLD18.2 polypeptide in an expressible form, and may optionally be included in one or more vectors.

In one example, the compositions and/or nucleic acid molecules of the application are provided with an instruction involving administration of the compositions or nucleic acid molecules to a subject suffering from or developing a tumor, pathogen, or immune disease. The instruction typically comprises information regarding the use of the composition for treating and/or preventing tumors or pathogenic infections. In one example, the instruction comprises at least one of the following: a description of the therapeutic agent: a dosage schedule and dosing for the treatment or prevention of tumors, pathogenic infections, or immune diseases, or symptoms thereof: precautions: warnings; indications; unindications; dosing information: adverse reactions: animal pharmacology: clinical studies; and/or references. These instructions may be printed directly on the container, or as a label affixed to the container, or provided as a separate sheet, booklet, card or folder within or with the container.

### Advantages of the Application:

1. The application significantly improves the efficacy of using immune effector cells that express chimeric antigen receptors for treating biliary tumors (including gallbladder cancer and cholangiocarcinoma): decrease of the number or volume of tumors, and/or reduction of the number and/or degree of metastasis, and/or decrease of tumor markers, and/or disappearance or weakening of common complications of mid- to late-stage cancer.
2. Significant anti-tumor effects on solid tumors have achieved, and the application provides a safe and effective treatment capable of stabilizing or even curing CLA18.2-positive mid-to-late biliary tumors (comprising gallbladder cancer and cholangiocarcinoma).
3. The application provides a pretreatment composition for improving the treatment effect of the immune effector cells against biliary tumors (including gallbladder cancer and cholangiocarcinoma). The fludarabine, cyclophosphamide, and albumin-bound paclitaxel composition are administrated before the immune effector cells, significantly promoting the anti-tumor efficacy of immune effector cells, and significantly reducing the tumor burden of subjects, thereby improving the quality of life of subjects and prolonging survival.

The application will be further described below in conjunction with specific examples. It should be understood that these examples are only used to illustrate the application, and are not intended to limit the scope of the application. In the following examples, the experimental methods without indicating the specific conditions are usually carried out according to conventional conditions, for example, those described in J. Sambrook et al., eds., Molecular Cloning Experimental Guide, 3rd Edition, Science Press, 2002, or as recommended by the manufacturer.

### Materials and Methods:

### Materials:

Various materials used in the application including reagents are commercial available.

Exemplary antigen receptors of the application, including CARs, and methods for engineering and introducing receptors into cells, referring to, for example, Chinese patent application publication numbers: CN107058354A, CN107460201A, CN105194661A, CN105315375A, CN105713881A, CN106146666A, CN106519037A, CN106554414A, CN105331585A, CN106397593A, CN106467573A, CN104140974A, CN 108884459A, CN107893052A, CN108866003A, CN108853144A, CN109385403A, CN109385400A, CN109468279A, CN109503715A, CN 109908176A, CN109880803A, CN 110055275A, CN110123837A, CN 110438082A, and CN 110468105A, and International patent application publication numbers: WO2017186121A1. WO2018006882A1, WO2015172339A8, WO2018/018958A1, WO2014180306A1, WO2015197016A1, WO2016008405A1, WO2016086813A1, WO2016150400A1, WO2017032293A1, WO2017080377A1, WO2017186121A1, WO2018045811A1, WO2018108106A1, WO 2018/219299, WO2018/210279, WO2019/024933, WO2019/114751, WO2019/114762, WO2019/141270, WO2019/149279, WO2019/170147A1, WO 2019/210863, and WO2019/219029.

### Example 1. Preparation of CAR-T Cells

Conventional molecular biology methods in the art are used to prepare CAR-T cells that express CARs specifically recognizing CLD18.2. Using PRRLSIN as a vector, the chimeric antigen receptor sequences shown in Table 1 were respectively inserted to construct lentiviral vectors that express the chimeric antigen receptor of the CLD18.2. The activated T cells were infected respectively, then culturing and expanding to the required quantity to obtain CLD18.2-BBZ CAR-T cells, CLD18.2-28Z CAR-T cells, and CLD18.2-28BBZ CAR-T cells.

TABLE 1
Chimeric Antigen Receptors

| Chimeric | |
|---|---|
| antigen | Signal peptide-extracellular binding region- |
| receptors | transmembrane region-intracellular signal region |
| CLD18.2-BBZ | CD8α signal peptide-CLD18.2-scFV-CD8 hinge-CD8 |
| | transmembrane region-CD137 intracellular domain, |
| | CD3ζ intracellular domain |
| CLD18.2-28Z | CD8α signal peptide-CLD18.2-scFV-CD8 hinge- |
| | CD28 transmembrane region-CD28 intracellular |
| | domain, CD3ζ intracellular domain |
| CLD18.2-28BBZ | CD8α signal peptide-CLD18.2-scFV-CD8 hinge- |
| | CD28 transmembrane region-CD28 intracellular |
| | domain, CD137 intracellular domain, CD3ζ |
| | intracellular domain |

Note:
CD8α signal peptide (SEQ ID NO: 1); CLDN18.2 scFV (SEQ ID NO: 2); CD8 hinge region (SEQ ID NO: 3); CD8 transmembrane region (SEQ ID NO: 4); CD28 transmembrane region (SEQ ID NO: 5); CD137 intracellular domain (SEQ ID NO: 6); CD28 intracellular domain (SEQ ID NO: 7); CD3ζ intracellular domain (SEQ ID NO: 8).

### Example 2. Cytotoxicity Assay of CAR-T Cells

The killing of CAR-T cells in vitro was detected by lactate dehydrogenase method. CytoTox 96® kit was used. For the specific steps, referring to the kit instructions.
(1) Target cells: human cholangiocarcinoma cell RBE and human gallbladder cancer cell GBC-SD that is negative for CLD18.2 expression; RBE-CLD18.2 cells and GBC-SD-CLD18.2 cells that exogenously express human CLD18.2.
(2) Effector cells: UTD (T cells that not infected with virus), CLD18.2-28Z CAR-T cells.

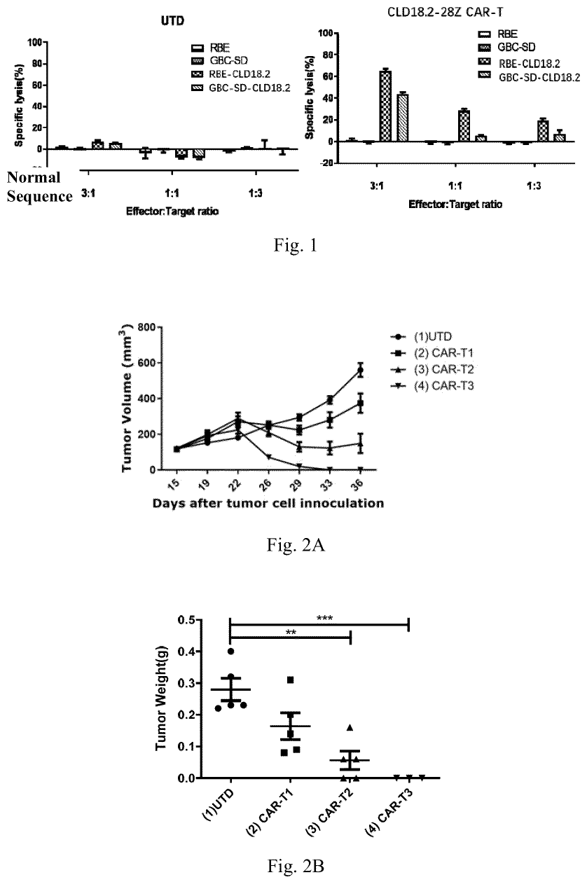

The cells were plated into 96-well plates according to the effect-to-target ratios of 3:1, 1:1, and 1:3, then co-culturing for 18 hours at 37° C. and 5% CO₂ for detection. As shown in FIG. 1: CLD18.2-28Z CAR-T cells killed CLD18.2-positive cells; CLD18.2-28Z CAR-T has basically no killing of CLD18.2-negative cells RBE and GBC-SD.

### Example 3. CAR-T Cell Killing Experiment In Vivo

3×10⁶ GBC-SD-CLD18.2 cells were inoculated subcutaneously into the right axilla of 5- to 6-week-old female NPG mice (purchased from Beijing Vitalstar Biotechnology Co., Ltd.), and the tumor-inoculated day was recorded as D0 (day 0). On D15 after inoculation, the tumor volumes grew to about 150 mm³, and cells were randomly divided into 4 groups, namely UTD group and CLD18.2-28Z CAR-T groups. The CLD18.2-28Z CAR-T groups included CAR-T1 (5*10\hat{ }5 cells/mouse) group, CAR-T2 (1*10\hat{ }6 cells/mouse) group, CAR-T3 (3*10\hat{ }6 cells/mouse) group. As shown in FIG. 2, on D36 after tumor cell inoculation, compared with UTD, the tumor inhibition rates of each group were: CAR-TI: 42.43%, CAR-T2: 80%; CAR-T3: 100% (Tumors of all 5 micewere regressed).

### Example 4. CAR-T Cell Therapy for Biliary Tract Tumors

Subjects with CLD18.2-positive biliary tumors were treated with CLD18A2-targeted CAR-T cells, and tumor tissue sections from previous surgeries or biopsies were used to detect CLD 18.2 expression. Before administrating CAR-T cells, subjects underwent apheresis technology called “mononuclear cell apheresis” and underwent pretreatment. Through apheresis, PBMC were obtained from the subjects, and CAR-T cells were obtained by transducing and amplifying a viral vector encoding an anti-CLD18A2 CAR (CLD18.2-28Z CAR was used as an exemplary CAR). Before administrating CAR-T cells, subjects were administrated pretreatment with: fludarabine, cyclophosphamide, and albumin-bound paclitaxel.

Prior to CAR-T therapy, tumor burden was optionally evaluated by measuring the size or shape of the solid tumors, for example, by PET or CT scan, and may also be evaluated by detecting tumor markers and/or observing the occurrence and severity of tumor complications.

When a subject requires at least one cycle of CAR-T treatment, the CAR-T in the subsequent cycle will be administrated 4 weeks after the completion of administration of CAR-T in the prior cycle. Each cycle of CAR-T administration may be administrated once or two or more times of intravenous (IV) infusions. The cells administrated each time were infused within about 3-30 minutes, preferably 5-25 minutes. After CAR-T administration in each cycle, subjects will receive a physical examination, and be monitored for any symptoms of toxicity or toxic outcomes, such as fever, hypotension, hypoxia, neurological deficits, or increase of inflammatory cytokines or C-reactive protein (CRP) serum levels. The examination may be performed by obtaining blood from the subject to evaluate the levels of cytokines indicative of CRS through ELISA, and/or MSD, and/or CBA. If necessary, anti-IL-6 therapy was administrated, or other CRS treatments were administrated, so as to reduce the symptoms of CRS. After the CAR-T administration was completed in each cycle, for example, 1, 2, 3, and/or 4 weeks after the start of the administration, the CAR-T quantity in the subject may be evaluated through qPCR, ELISA, ELISPOT, antibody assay, etc.

The reduction in tumor burden after each cycle of treatment may be measured by scanning (e.g., PET and CT scans), and/or by quantifying cells that are positive for antigens (e.g., claudin 18.2) in the blood or tumor site.

Exemplary Cases, subject 1: male, 65 years old, 44 kg, poorly differentiated adenocarcinoma of the gallbladder, CLD18.2 expression was +++ (strong positive staining), and the percentage of stained tumor cells was 90%. He had previously undergone palliative cholecystectomy, fluorouracil and other chemotherapy drugs, and had distant metastasis. Pretreatment was performed before CAR-T treatment: fludarabine is 35 mg/d on Day 1-2, cyclophosphamide is 350 mg/d on Day 1-3, and albumin-bound paclitaxel is 100 mg/d on Day 2. The subject received 2 cycles of CAR-T infusion, the infusion dose in cycle 1 was 3.75×10⁸ cells, and the infusion dose in cycle 2 was 2.5×10⁸ cells, both of which were one-time reinfusions.

Subject 2: female, 63 years old, 59 kg, with high and middle differentiated adenocarcinoma of the gallbladder, CLD18.2 expression was +++ (strong positive staining), and the percentage of stained tumor cells was 80%. She had previously undergone cholecystectomy, albumin-bound paclitaxel, S-1 and other chemotherapy drugs, and pembrolizumab immunotherapy, and had distant metastasis. Pretreatment was performed before CAR-T treatment: fludarabine is 38.75 mg/d on Day 1-2, cyclophosphamide is 387.5 mg/d on Day 1-3, and albumin-bound paclitaxel is 100 mg/d on Day 2. The subject received 1 cycle of CAR-T infusion, and the infusion dose was 2.5×10⁸ cells as a one-time reinfusion.

Subject 3: Male, 57 years old, 65 kg, moderately to poorly differentiated carcinoma of the extrahepatic bile duct, CLD18.2 expression was +++ (strong positive staining), and the percentage of stained tumor cells was 80%. He had previously undergone pancreaticoduodenectomy, treatment with gemcitabine, raltitrexed, cisplatin and other chemotherapy drugs, as well as PD-L1/TGF-β dual antibody (Bintrafusp alfa) or placebo treatment, and currently had distant metastasis. Pretreatment was performed before CAR-T treatment: fludarabine is 42.25 mg/d on Day 1-2, cyclophosphamide is 422.5 mg/d on Day 1-3, and albumin-bound paclitaxel is 100 mg/d on Day 2. The subject received 1 cycle of CAR-T cell infusion, and the infusion dose was 2.5×10⁸ cells as a one-time reinfusion.

### Example 5: Evaluation of Treatment

The tumor burden before and after treatment was evaluated through imaging detection of target lesions and non-target lesions, and the number and size of target lesions were determined. For the efficacy evaluation criteria, please refer to the Efficacy Evaluation Criteria for Solid Tumors (version 1.1). All adverse events (AE) will be classified according to the coding of the latest version of the ICH International Dictionary of Medical Terminology MedDRA, and analyzed according to the Common Standard Terminology Criteria for Adverse Events (CTCAE v5.0 version, classification). Particularly, cytokine release syndrome (CRS) and neurotoxicity were graded and treated according to the ASTCT consensus.

The three subjects with biliary tract tumor did not suffer from severe CRS or neurotoxicity, the CAR-T therapy targeting CLD18.2 is safe. Particularly two subjects with gallbladder cancer developed grade 1-2 CRS after CAR-T reinfusion, the main clinical manifestations were fever, tachycardia, and peripheral edema: hypotension and hypoxemia may also be seen, and they were recovered after symptomatic and supportive treatment. One subject with cholangiocarcinoma did not develop CRS after CAR-T reinfusion. Before and after CAR-T treatment, the weight of the three subjects remained stable.

Subject 1 is in PR after treatment, with a PFS of 4.2 months, and an OS of approximately 10 months. Subjects 2 and 3 reached SD after treatment, with disease progression-free survival periods of approximately 9.3 months and 7.5 months respectively, and both are still in the stable phase.

The continued survival period of CAR-T cells in the body was detected, that is, the period during which CAR-T cells continue to survive after being “implanted” in the body. From the end of the initial infusion (Day 0), the Q-PCR method was used at each visit point (probe (SEQ ID NO: 20), upstream primer (SEQ ID NO: 21), downstream primer (SEQ ID NO: 22) were used) to detect the copy number of CAR-CLD18 DNAs contained in peripheral blood. The results showed that after CAR-T infusion, the amplified copy number on D5 (Day 5) in subject 1 reached a peak of 6713 copies/μg gDNA, and persisted for at least 62 days: in subject 2, the amplified copy number reached a peak of 8237 copies/μg gDNA on D7, and persisted for at least 85 days; in subject 3, the amplified copy number reached a peak value of 5698 copies/μg gDNA on D14, and persisted for at least 28 days.

All documents mentioned in the application are herein incorporated by reference to the same extent, as if each individual document was individually incorporated by reference. In addition, it should be understood that after reading the above content of the application, those skilled in the art can make various changes or modifications to the application, and these equivalent forms also fall within the scope defined by the appended claims of the application.

The sequences used in the application are listed in the table below:

| SEQ ID | Name in the | |
|---|---|---|
| NO. | application | Sequences |
| 1 | CD8α | MALPVTALLLPLALLLHAARP |
| | signal | |
| | peptide | |
| 2 | CLDN18.2- | QVQLQESGPGLIKPSQTLSLTCTVSGGSISSGYNWHW |
| | scFv | IRQPPGKGLEWIGYIHYTGSTNYNPALRSRVTISVDTS |
| | | KNQFSLKLSSVTAADTAIYYCARIYNGNSFPYWGQG |
| | | TTVTVSSGGGGSGGGGSGGGGSDIVMTQSPDSLAVS |
| | | LGERATINCKSSQSLFNSGNQKNYLTWYQQKPGQPP |
| | | KLLIYWASTRESGVPDRFSGSGSGTDFTLTISSLQAED |
| | | VAVYYCQNAYSFPYTFGGGTKLEIKR |
| 3 | CD8 | TTTPAPRPPTPAPTIASQPLSLRPEACRPAAGGAVHTR |
| | hinge | GLDFACD |
| | region | |
| 4 | CD8 | IYIWAPLAGTCGVLLLSLVITLYC |
| | transmembrane | |
| | region | |
| 5 | CD28 | FWVLVVVGGVLACYSLLVTVAFIIFWV |
| | transmembrane | |
| | region | |
| 6 | CD137 | KRGRKKLLYIFKQPFMRPVQTTQEEDGCSCRFPEEEE |
| | intracellular | GGCEL |
| | domain | |
| 7 | CD28 | RSKRSRLLHSDYMNMTPRRPGPTRKHYQPYAPPRDF |
| | intracellular | AAYRS |
| | domain | |
| 8 | CD32 | RVKFSRSADAPAYQQGQNQLYNELNLGRREEYDVL |
| | intracellular | DKRRGRDPEMGGKPQRRKNPQEGLYNELQKDKMAE |
| | domain | AYSEIGMKGERRRGKGHDGLYQGLSTATKDTYDAL |
| | | HMQALPPR |
| 9 | Human | MAVTACQGLGFVVSLIGIAGIIAATCMDQWSTQDLY |
| | CLDN18.2 | NNPVTAVFNYQGLWRSCVRESSGFTECRGYFTLLGL |
| | | PAMLQAVRALMIVGIVLGAIGLLVSIFALKCIRIGSME |
| | | DSAKANMTLTSGIMFIVSGLCAIAGVSVFANMLVTNF |
| | | WMSTANMYTGMGGMVQTVQTRYTFGAALFVGWV |
| | | AGGLTLIGGVMMCIACRGLAPEETNYKAVSYHASGH |
| | | SVAYKPGGFKASTGFGSNTKNKKIYDGGARTEDEVQ |
| | | SYPSKHDYV |
| 10 | Amino | QVQLQESGPGLIKPSQTLSLTCTVSGGSISSGYNWH |
| | acids of | WIRQPPGKGLEWIGYIHYTGSTNYNPALRSRVTISV |
| | CLDN18.2- | DTSKNQFSLKLSSVTAADTAIYYCARIYNGNSFPY |
| | 1-VH | WGQGTTVTVSS |
| 11 | Nucleic | caggtgcagctgcaggagagcggccccggcctgatcaagcccagccagacc |
| | acid of | ctgagcctgacctgcaccgtgagcggcggcagcatcagcagcggctacaact |
| | CLDN18.2- | ggcactggatccggcagccccccggcaagggcctggagtggatcggctaca |
| | 1-VH | tccactacaccggcagcaccaactacaaccccgccctgcggagccgggtga |
| | | ccatcagcgtggacaccagcaagaaccagttcagcctgaagctgagcagcgt |
| | | gaccgccgccgacaccgccatctactactgcgcccggatctacaacggcaac |
| | | agcttcccctactggggccagggcaccaccgtgaccgtgagcagc |
| 12 | Amino | DIVMTQSPDSLAVSLGERATINCKSSQSLFNSGNQK |
| | acids of | NYLTWYQQKPGQPPKLLIYWASTRESGVPDRFSGS |
| | CLDN18.2- | GSGTDFTLTISSLQAEDVAVYYCQNAYSFPYTFGG |
| | 1-VL | GTKLEIKR |
| 13 | Nucleic | gacatcgtgatgacccagagccccgacagcctggccgtgagcctgggcgag |
| | acid of | cgggccaccatcaactgcaagagcagccagagcctgttcaacagcggcaac |
| | CLDN18.2- | cagaagaactacctgacctggtaccagcagaagcceggccagccccccaag |
| | 1-VL | ctgctgatctactgggccagcacccgggagagcggcgtgcccgaccggttca |
| | | gcggcagcggcagcggcaccgacttcaccctgaccatcagcagcctgcagg |
| | | ccgaggacgtggccgtgtactactgccagaacgcctacagcttcccctacacc |
| | | ttcggcggcggcaccaagctggagatcaagcgg |
| 14 | HCDR1 | SGYNWH |
| 15 | HCDR2 | YIHYTGSTNYNPALRS |
| 16 | HCDR3 | IYNGNSFPY |
| 17 | LCDR1 | KSSQSLFNSGNQKNYLT |
| 18 | LCDR2 | WASTRES |
| 19 | LCDR3 | QNAYSFPYT |
| 20 | Probe | CTGAGCAGCGTGACCGCCGC |
| 21 | Upstream | TGGAGTGGATCGGCTACATC |
| | primer | |
| 22 | Downstream | AGTAGTAGATGGCGGTGTCG |
| | primer | |
| 23 | Antibody 2- | qvqlqqsgaelarpgasvkmsckasgytftsytmhwvkqrpgqglewigyin |
| | scFv | pssgytnynqkfkdkatltadkssstaymqlssltsedsavyycariyygnsfay |
| | | wgqgttvtvssggggsggggsggggsdivmtqspssltvtagekvtmsckssq |
| | | sllnsgnqknyltwyqqkpgqppklliywastresgvpdrftgsgsgtdftltissv |
| | | qaedlavyycqndysypltfgagtklelkr |
| 24 | Antibody 3- | QVQLQQSGPELVKPGALVKISCKASGYTFTSYDIN |
| | scFv | WVKQRPGQGLEWIGWIYPGDGSTKYNEKFKGKAT |
| | | LTADKSSSTAYMQLSSLTSENSAVYFCARGGYRYD |
| | | EAMDYWGQGTTVTVSSggggsggggsggggsQIVLTQSP |
| | | AIMSASPGEKVTMTCSASSSISYMHWYQQKPGTSP |
| | | KRWIYDTSKLASGVPARFSGSGSGTSYSLTISSMEA |
| | | EDAATYYCHQRSSYPYTFGGGTKLEIKR |
| 25 | Antibody4- | qiqlvqsgpelkkpgetvkisckasgytftnygmnwvkqapgkglkwmg |
| | scFv | wintntgeptyaeefkgrfafsletsastaylqinnlknedtatyfcarfsygns |
| | | faywgqgttvtvssggggsggggsggggsdivmtqspsslsvsagekvtms |
| | | ckssqsllnsgnqknylawyqqkpgqppklliygastresgvpdrftgsgsg |
| | | tdftltissvqaedlavyycqndhsypltfgagtklelkr |
| 26 | Antibody 5- | dvqlqesgpdlvkpsqslsltctvtgysitsgynwhwirqfpgnkmewmg |
| | scFv | yihytgstnynpslrsrisitrdtsknqfflqlnsvttddtatyyctriyngnsfp |
| | | ywgqgtsvtvssggggsggggsggggsdivmtqspssltvtpgekvtmtck |
| | | ssqslfnsgnqknyltwyqqrpgqppkmliywastresgvpdrftgsgsgt |
| | | dftltissvqaedlavfycqnaysfpytfgggtkleikr |
| 27 | Antibody6- | qvqlqqsgaelarpgasvkmsckasgytftsytmhwvkqrpgqglewig |
| | scFv | yidpssgytnynqkfkdkatltadkssstaymqlssltsedsavyycariyyg |
| | | nsfaywgqgttvtvssggggggggsggggsdivmtqspssltvtagekvt |
| | | msckssqsllnsgnqknyltwyqqkpgqppklliywastresgvpdrftgs |
| | | gsgtdftltissvqaedlavyycqndysypltfgagtklelkr |
| 28 | Antibody 7- | qvqlqqsgaelarpgasvkmsckasgytftsytmhwvkqrpgqglewig |
| | scFv | yinpasgytnynqkfkdkatltadkssstaymqlssltsedsavyycariyyg |
| | | nsfaywgqgttvtvssggggsggggsggggsdivmtqspssltvtagekvt |
| | | msckssqsllnsgnqknyltwyqqkpgqppklliywastresgvpdrftgs |
| | | gsgtdftltissvqaedlavyycqndysypltfgagtklelkr |
| 29 | Antibody 8- | qvqlvqsgaevkkpgasvkvsckasgytftsytmhwvrqapgqglewm |
| | scFv | gyinpasgytnynqkfkdrvtmtrdtststaymelsslrsedtavyycariyy |
| | | gnsfaywgqgtlvtvssggggsggggsggggsdivmtqspdslavslgerat |
| | | inckssqsllnsgnqknyltwyqqkpgqppklliywastresgvpdrfsgsg |
| | | sgtdftltisslqaedvavyycqndysypltfgggtkveikr |
| 30 | Antibody9- | qvqlqesgpglvkpsqtlsltctvsggsissgynwhwirqppgkglewigyi |
| | scFv | hytgstnynpalrsrvtisvdtsknqfslklssvtaadtavyycariyngnsfp |
| | | ywgqgttvtvssggggsggggsggggsdivmtqspdslavslgeratincks |
| | | sqslfnsgnqknyltwyqqkpgqppklliywastresgvpdrfsgsgsgtdf |
| | | tltisslqaedvavyycqnaysfpytfgggtkleikr |
| 31 | Antibody 10- | qiqlvqsgpelkkpgetvkisckasgytftnygmnwvkqapgkglkwmg |
| | scFv | wintntgeptyaeefkgrfafsletsastaylqinnlknedtatyfcarlgfgna |
| | | mdywgqgtsvtvssggggsggggsggggsdivmtqspssltvtagekvt |
| | | msckssqsllnsgnqknyltwyqqkpgqppklliywastresgvpdrftgs |
| | | gsgtdftltissvqaedlavyycqndysypltfgagtklelkr |
| 32 | Antibody 11- | qvqlqqpgaelvrpgasvklsckasgytftsywinwvkqrpgqglewigni |
| | scFv | ypsdsytnynqkfkdkatltvdkssstaymqlssptsedsavyyctrswrgn |
| | | sfdywgqgttltvssggggsggggggggsdivmtqspssltvtagekvtm |
| | | sckssqsllnsgnqknyltwyqqkpgqppklliywastresgvpdrftgsgs |
| | | gtdftltissvqaedlavyycqndysypftfgsgtkleikr |
| 33 | Antibody 12- | QVQLQQPGAELVRPGASVKLSCKASGYTFTSYWIN |
| | scFv | WVKQRPGQGLEWIGNIYPSDSYTNYNQKFKDKAT |
| | | LTVDKSSSTAYMQLSSPTSEDSAVYYCTRSWRGNS |
| | | FDYWGQGTTLTVSSGGGGSGGGGSGGGGSDIVMT |
| | | QSPSSLTVTAGEKVTMSCKSSQSLLNSGNQKNYLT |
| | | WYQQKPGQPPKLLIYWASTRESGVPDRFTGSGSGT |
| | | DFTLTISSVQAEDLAVYYCONDYSYPFTFGSGTKLE |
| | | IK |
| 34 | 28Z | TTTPAPRPPTPAPTIASQPLSLRPEACRPAAGGAVHT |
| | | RGLDFACDFWVLVVVGGVLACYSLLVTVAFIIFWV |
| | | RSKRSRLLHSDYMNMTPRRPGPTRKHYQPYAPPRD |
| | | FAAYRSRVKFSRSADAPAYQQGQNQLYNELNLGR |
| | | REEYDVLDKRRGRDPEMGGKPQRRKNPQEGLYNE |
| | | LQKDKMAEAYSEIGMKGERRRGKGHDGLYQGLST |
| | | ATKDTYDALHMQALPPR |
| 35 | BBZ | TTTPAPRPPTPAPTIASQPLSLRPEACRPAAGGAVHT |
| | | RGLDFACDIYIWAPLAGTCGVLLLSLVITLYCKRGR |
| | | KKLLYIFKQPFMRPVQTTQEEDGCSCRFPEEEEGGC |
| | | ELRVKFSRSADAPAYQQGQNQLYNELNLGRREEY |
| | | DVLDKRRGRDPEMGGKPQRRKNPQEGLYNELQKD |
| | | KMAEAYSEIGMKGERRRGKGHDGLYQGLSTATKD |
| | | TYDALHMQALPPR |
| 36 | 28BBZ | TTTPAPRPPTPAPTIASQPLSLRPEACRPAAGGAVHT |
| | | RGLDFACDFWVLVVVGGVLACYSLLVTVAFIIFWV |
| | | RSKRSRLLHSDYMNMTPRRPGPTRKHYQPYAPPRD |
| | | FAAYRSKRGRKKLLYIFKQPFMRPVQTTQEEDGCS |
| | | CRFPEEEEGGCELRVKFSRSADAPAYQQGQNQLYN |
| | | ELNLGRREEYDVLDKRRGRDPEMGGKPQRRKNPQ |
| | | EGLYNELQKDKMAEAYSEIGMKGERRRGKGHDGL |
| | | YQGLSTATKDTYDALHMQALPPR |
| 37 | CLDN18.2- | caggtgcagctgcaggagagcggccccggcctgatcaagcccagccagacc |
| | 28Z | ctgagcctgacctgcaccgtgagcggcggcagcatcagcagcggctacaact |
| | | ggcactggatccggcagccccccggcaagggcctggagtggatcggctaca |
| | | tccactacaccggcagcaccaactacaaccccgccctgcggagccgggtga |
| | | ccatcagcgtggacaccagcaagaaccagttcagcctgaagctgagcagcgt |
| | | gaccgccgccgacaccgccatctactactgcgcccggatctacaacggcaac |
| | | agcttcccctactggggccagggcaccaccgtgaccgtgagcagcggtgga |
| | | ggcggttcaggcggaggtggttctggcggtggcggatcggacatcgtgatga |
| | | cccagagccccgacagcctggccgtgagcctgggcgagcgggccaccatca |
| | | actgcaagagcagccagagcctgttcaacagcggcaaccagaagaactacct |
| | | gacctggtaccagcagaagcccggccagccccccaagctgctgatctactgg |
| | | gccagcacccgggagagcggcgtgcccgaccggttcagcggcagcggcag |
| | | cggcaccgacttcaccctgaccatcagcagcctgcaggccgaggacgtggc |
| | | cgtgtactactgccagaacgcctacagcttcccctacaccttcggcggcggca |
| | | ccaagctggagatcaagcggaccacgacgccagcgccgcgaccaccaaca |
| | | ccggcgcccaccatcgcgtcgcagcccctgtccctgcgcccagaggcgtgc |
| | | cggccagcggcggggggcgcagtgcacacgagggggctggacttcgcctg |
| | | tgatttttgggtgctggtggtggttggtggagtcctggcttgctatagcttgctagt |
| | | aacagtggcctttattattttctgggtgaggagtaagaggagcaggctcctgca |
| | | cagtgactacatgaacatgactccccgccgccccgggccaacccgcaagcat |
| | | taccagccctatgccccaccacgcgacttcgcagcctatcgctccagagtgaa |
| | | gttcagcaggagcgcagacgcccccgcgtaccagcagggccagaaccagct |
| | | ctataacgagctcaatctaggacgaagagaggagtacgatgttttggacaaga |
| | | gacgtggccgggaccctgagatggggggaaagccgcagagaaggaagaac |
| | | cctcaggaaggcctgtacaatgaactgcagaaagataagatggcggaggcct |
| | | acagtgagattgggatgaaaggcgagcgccggaggggcaaggggcacgat |
| | | ggcctttaccagggtctcagtacagccaccaaggacacctacgacgcccttca |
| | | catgcaggccctgccccctcgc |
| 38 | CLDN18.2- | caggtgcagctgcaggagagcggccccggcctgatcaagcccagccagacc |
| | BBZ | ctgagcctgacctgcaccgtgagcggcggcagcatcagcagcggctacaact |
| | | ggcactggatccggcagccccccggcaagggcctggagtggatcggctaca |
| | | tccactacaccggcagcaccaactacaaccccgccctgcggagccgggtga |
| | | ccatcagcgtggacaccagcaagaaccagttcagcctgaagctgagcagcgt |
| | | gaccgccgccgacaccgccatctactactgcgcccggatctacaacggcaac |
| | | agcttcccctactggggccagggcaccaccgtgaccgtgagcagcggtgga |
| | | ggcggttcaggcggaggtggttctggcggtggcggatcggacatcgtgatga |
| | | cccagagccccgacagcctggccgtgagcctgggcgagcgggccaccatca |
| | | actgcaagagcagccagagcctgttcaacagcggcaaccagaagaactacct |
| | | gacctggtaccagcagaagcccggccagccccccaagctgctgatctactgg |
| | | gccagcacccgggagagcggcgtgcccgaccggttcagcggcagcggcag |
| | | cggcaccgacttcaccctgaccatcagcagcctgcaggccgaggacgtggc |
| | | cgtgtactactgccagaacgcctacagcttcccctacaccttcggcggcggca |
| | | ccaagctggagatcaagcggaccacgacgccagcgccgcgaccaccaaca |
| | | ccggcgcccaccatcgcgtcgcagcccctgtccctgcgcccagaggcgtgc |
| | | cggccagcggcggggggcgcagtgcacacgagggggctggacttcgcctg |
| | | tgatatctacatctgggcgcccttggccgggacttgtggggtccttctcctgtca |
| | | ctggttatcaccctttactgcaaacggggcagaaagaaactcctgtatatattca |
| | | aacaaccatttatgagaccagtacaaactactcaagaggaagatggctgtagct |
| | | gccgatttccagaagaagaagaaggaggatgtgaactgagagtgaagttcag |
| | | caggagcgcagacgcccccgcgtaccagcagggccagaaccagctctataa |
| | | cgagctcaatctaggacgaagagaggagtacgatgttttggacaagagacgtg |
| | | gccgggaccctgagatggggggaaagccgcagagaaggaagaaccctcag |
| | | gaaggcctgtacaatgaactgcagaaagataagatggcggaggcctacagtg |
| | | agattgggatgaaaggcgagcgccggaggggcaaggggcacgatggccttt |
| | | accagggtctcagtacagccaccaaggacacctacgacgcccttcacatgca |
| | | ggccctgccccctcgc |
| 39 | CLDN18.2- | caggtgcagctgcaggagagcggccccggcctgatcaagcccagccagacc |
| | 28BBZ | ctgagcctgacctgcaccgtgagcggcggcagcatcagcagcggctacaact |
| | | ggcactggatccggcagccccccggcaagggcctggagtggatcggctaca |
| | | tccactacaccggcagcaccaactacaaccccgccctgcggagccgggtga |
| | | ccatcagcgtggacaccagcaagaaccagttcagcctgaagctgagcagcgt |
| | | gaccgccgccgacaccgccatctactactgcgcccggatctacaacggcaac |
| | | agcttcccctactggggccagggcaccaccgtgaccgtgagcagcggtgga |
| | | ggcggttcaggcggaggtggttctggcggtggcggatcggacatcgtgatga |
| | | cccagagccccgacagcctggccgtgagcctgggcgagcgggccaccatca |
| | | actgcaagagcagccagagcctgttcaacagcggcaaccagaagaactacct |
| | | gacctggtaccagcagaagcccggccagccccccaagctgctgatctactgg |
| | | gccagcacccgggagagcggcgtgcccgaccggttcagcggcagcggcag |
| | | cggcaccgacttcaccctgaccatcagcagcctgcaggccgaggacgtggc |
| | | cgtgtactactgccagaacgcctacagcttcccctacaccttcggcggcggca |
| | | ccaagctggagatcaagcggaccacgacgccagcgccgcgaccaccaaca |
| | | ccggcgcccaccatcgcgtcgcagcccctgtccctgcgcccagaggcgtgc |
| | | cggccagcggcggggggcgcagtgcacacgagggggctggacttcgcctg |
| | | tgatttttgggtgctggtggtggttggtggagtcctggcttgctatagcttgctagt |
| | | aacagtggcctttattattttctgggtgaggagtaagaggagcaggctcctgca |
| | | cagtgactacatgaacatgactccccgccgccccgggccaacccgcaagcat |
| | | taccagccctatgccccaccacgcgacttcgcagcctatcgctccaaacgggg |
| | | cagaaagaaactcctgtatatattcaaacaaccatttatgagaccagtacaaact |
| | | actcaagaggaagatggctgtagctgccgatttccagaagaagaagaaggag |
| | | gatgtgaactgagagtgaagttcagcaggagcgcagacgcccccgcgtacca |
| | | gcagggccagaaccagctctataacgagctcaatctaggacgaagagagga |
| | | gtacgatgttttggacaagagacgtggccgggaccctgagatggggggaaag |
| | | ccgcagagaaggaagaaccctcaggaaggcctgtacaatgaactgcagaaa |
| | | gataagatggcggaggcctacagtgagattgggatgaaaggcgagcgccgg |
| | | aggggcaaggggcacgatggcctttaccagggtctcagtacagccaccaag |
| | | gacacctacgacgcccttcacatgcaggccctgccccctcgc |

1. A method for treating a subject suffering from or suspected of suffering from a CLD18 positive biliary tract tumor, comprising administrating cells that express exogenous receptors targeting CLD18 to the subject.

2. The method according to claim 1, wherein the CLD18 is CLD18.2.

3. The method according to claim 1, wherein the biliary tract tumor comprises a gallbladder cancer and a cholangiocarcinoma.

4. The method according to claim 1, wherein the cells are immune effector cells, preferably the immune effector cells are selected from the group consisting of: T cells, NK cells, NKT cells, mastocytes, macrophages, dendritic cells, CIK cells, stem cell-derived immune effector cells, or a combination thereof; further preferably the immune effector cells are derived from natural T cells and/or T cells induced by pluripotent stem cells.

5. The method according to claim 4, wherein the immune effector cells are autologous or allogeneic T cells, or primary T cells.

6. The method according to claim 1, wherein the exogenous receptors are selected from the group consisting of: chimeric antigen receptor (CAR), T cell receptor (TCR), T cell fusion protein (TFP), T cell antigen coupler (TAC), antibody-TCR chimera, or a combination thereof; and the antigen binding domains of the exogenous receptors specifically bind to CLD18.2.

7. The method according to claim 6, wherein the CAR comprises:
(i) an antibody specifically binding to CLD18.2, a transmembrane region of CD8 or CD28, and a intracellular domain of CD3ζ;
(ii) an antibody specifically binding to CLD18.2, a transmembrane region of CD8 or CD28, a intracellular domain of CD137, and a intracellular domain of CD3ζ;
(iii) an antibody specifically binding to CLD18.2, a transmembrane region of CD8 or CD28, a intracellular domain of CD28, and a intracellular domain of CD3ζ; or
(iv) an antibody specifically binding to CLD18.2, a transmembrane region of CD8 or CD28, a intracellular domain of CD28, a intracellular domain of CD137, and a intracellular domain of CD3ζ.

8. The method according to claim 1, wherein the cells are administrated to the subject for at least one cycle for treatment, preferably the cells are administrated to the subject for 1-3 cycles for treatment.

9. The method according to claim 8, wherein a dose of the cells in a cell therapy product administrated per cycle is no more than: about 2×10⁹ cells/kg of subject body weight, about 2×10⁸ cells/kg of subject body weight, or about 2×10⁷ cells/kg of subject body weight; or
a dose of the cells in a cell therapy product is no more than about 1×10¹¹ cells/subject, about 1×10¹⁰ cells/subject, about 5×10⁹ cells/subject, about 2×10⁹ cells/subject, or about 1×10⁹ cells/subject.

10. The method according to claim 9, wherein a dose of the cells in a cell therapy product administrated per cycle is about 1×10⁵ cells/kg of subject body weight to 2×10⁷ cells/kg of subject body weight, or about 1×10⁶ cells/kg of subject body weight to 2×10⁷ cells/kg of subject body weight; or
a dose of the cells in a cell therapy product administrated per cycle is about 1×10⁷ cells to 5×10⁹ cells/subject, about 1×10⁷ cells/subject to 2×10⁹ cells/subject, or about 1×10⁷ cells/subject to 1×10⁹ cells/subject; or
a dose of the cells in a cell therapy product administrated per cycle is about 1×10⁸ cells/subject to 5×10⁹ cells/subject, about 1×10⁸ cells/subject to 2×10⁹ cells/subject, or from about 1×10⁸ cells to 1×10⁹ cells/subject; or
a dose of the cells in a cell therapy product administrated per cycle is about 2.5×10⁸ cells/subject to 5×10⁸ cells/subject.

11. The method according to claim 8, wherein a pretreatment is performed before administrating the cell therapy product in each cycle, and the pretreatment includes administrating chemical drug, biological drug, radiotherapy or a combination thereof to the subject.

12. The method according to claim 11, wherein the pretreatment is performed 1-8 days before administrating the cell therapy product; preferably 2-6 days before administrating the cell therapy product; and preferably each of the chemical drug, the biological drug, the radiotherapy or a combination thereof is used continuously for no more than 4 days.

13. The method according to claim 11, wherein the chemical drug is any one or at least two selected from the group consisting of: cyclophosphamide, fludarabine, tubulin inhibitor, and pyrimidine anti-tumor drug; or the chemical drug comprises cyclophosphamide and fludarabine; or the chemical drug comprises cyclophosphamide, fludarabine, and tubulin inhibitor.

14. The method according to claim 13, wherein the tubulin inhibitor comprises taxane compound; or the tubulin inhibitor comprises paclitaxel, albumin-bound paclitaxel, and docetaxel; or the tubulin inhibitor is albumin-bound paclitaxel.

15. The method according to claim 13, wherein the dosage of fludarabine is about 10-50 mg/m²/day, or about 15-40 mg/m²/day, or about 15-30 mg/m²/day, or about 20-30 mg/m²/day; or about 25 mg/m²/day, or about 30-60 mg/day, or about 30-50 mg/day, or about 35-45 mg/day; or
the dosage of cyclophosphamide is about 200-400 mg/m²/day, or about 200-300 mg/m²/day, or about 250 mg/m²/day, or about 300-1000 mg/day, or about 300-550 mg/day, or about 300-500 mg/day; or
the dosage of taxane compound is no more than about 300 mg/day, or no more than about 200 mg/day, or about 90-120 mg/day.

16. The method according to claim 13, wherein the cyclophosphamide is administrated 2-3 times; or the fludarabine is administrated 1-2 times; or the taxane compound is administrated once.

17. The method according to claim 6, wherein the antigen binding domain or the antibody has a scFv sequence represented by SEQ ID NOs: 2, 23, 24, 25, 26, 27, 28, 29, 30, 31, 32 or 33; or
the antigen binding domain or the antibody has a HCDR1 represented by SEQ ID NO: 14, a HCDR2 represented by SEQ ID NO: 15, and a HCDR3 represented by SEQ ID NO: 16, a LCDR1 represented by SEQ ID NO: 17, a LCDR2 represented by SEQ ID NO: 18, and a LCDR3 represented by SEQ ID NO: 19; or
the antigen binding domain or the antibody has a heavy chain variable region represented by SEQ ID NO: 10 and a light chain variable region represented by SEQ ID NO: 12.

18. The method according to claim 6, wherein the chimeric antigen receptor has a scFv sequence represented by SEQ ID NOs: 2, 23, 24, 25, 26, 27, 28, 29, 30, 31, 32 or 33 which is sequentially connected to SEQ ID NOs: 34, 35 or 36 respectively; or the chimeric antigen receptor has a nucleotide sequence represented by SEQ ID NOs: 37, 38 or 39.

19-20. (canceled)

21. Use of cells that express exogenous receptors targeting CLD18 for treating a subject suffering from or suspected of suffering from CLD18 positive biliary tumor.

22. Use of cells that express exogenous receptors targeting CLD18 and chemical drugs or other biological drugs or radiotherapy for treating a subject suffering from or suspected of suffering from CLD18 positive biliary tumor.
